(19) United States
(12) Patent Application Publication
(10) Pub. No.: US 2025/0102509 A1
(43) Pub. Date: Mar. 27, 2025

(54) METHODS AND SYSTEMS FOR DETERMINING LEUKEMIA OR LYMPHOMA LEVELS USING LYMPHOID IMAGES

(71) Applicant: BOARD OF REGENTS, THE UNIVERSITY OF TEXAS SYSTEM, Austin, TX (US)

(72) Inventors: Joseph KHOURY, Houston, TX (US); Jia WU, Houston, TX (US); Siba EL HUSSEIN, Houston, TX (US); Pingjun CHEN, Houston, TX (US)

(21) Appl. No.: 18/728,323

(22) PCT Filed: Jan. 13, 2023
(86) PCT No.: PCT/US2023/060636

§ 371 Date: Jul. 11, 2024

Related U.S. Application Data
(60) Provisional application No. 63/299,554, filed on Jan. 14, 2022.
(60) Provisional application No. 63/364,233, filed on May 5, 2022.

(51) Int. Cl. G01N 33/574; G06V 10/44; G06V 10/762; G06V 10/764; G06V 10/82; G06V 20/69

(52) U.S. Cl. CPC G01N 33/57426; G06V 10/454; G06V 10/762; G06V 10/764; G06V 10/82; G06V 20/695

(57) ABSTRACT

Methods and systems that determine levels of leukemia or lymphoma are described. A model using biomarkers such as nuclear size, nuclear intensity, cellular density, and cell-to-nearest-neighbor distance may distinguish samples with chronic lymphocytic leukemia (CLL) progression, accelerated CLL (aCLL), and transformation to diffuse large B-cell lymphoma (Richter transformation; RT). Additionally, using a heat value based on nuclear size and nuclear intensity allowed for visualization or identification of proliferation centers (PCs) and also could be used to diagnose tissue samples from subjects with CLL. Some embodiments use unsupervised clustering to divide lymphoid cells into different cell types, and feature values of these cell types can be used to classify disorders. Methods and systems may accurately and efficiently diagnose levels of a disorder. Diagnosis may be performed with less biological sample (e.g., from a core-needle biopsy), resulting in a more rapid and minimally invasive biopsy.

### CROSS-REFERENCES TO RELATED APPLICATIONS

The present application claims the benefit of priority to U.S. Provisional Patent Application 63/299,554, filed on Jan. 14, 2022, and U.S. Provisional Patent Application No. 63/364,233, filed on May 5, 2022, the entire contents of both of which are incorporated herein by reference for all purposes.

### STATEMENT AS TO RIGHTS TO INVENTIONS MADE UNDER FEDERALLY SPONSORED RESEARCH AND DEVELOPMENT

This invention was made with government support under CA218667 awarded by the National Institutes of Health. The government has certain rights in the invention.

### FIELD

The present application relates to methods of diagnosing progression of disorders in images. Specifically, the present application relates to using biomarkers to diagnose progression of chronic lymphocytic leukemia (CLL) in lymphoid images.

### BACKGROUND

Artificial intelligence-based tools designed to assist in the diagnosis of lymphoid neoplasms remain limited. The development of such tools can add value as a diagnostic aid in the evaluation of tissue samples involved by lymphoma. A common diagnostic question is the determination of chronic lymphocytic leukemia (CLL) progression to accelerated CLL (aCLL) or transformation to diffuse large B-cell lymphoma (Richter transformation; RT) in subjects who develop aggressive disease. The morphologic assessment of CLL, aCLL, and RT can be diagnostically challenging.

### SUMMARY

Using established diagnostic criteria of CLL progression/transformation, we designed four artificial intelligence-constructed biomarkers based on cytologic (nuclear size and nuclear intensity) and architectural (cellular density and cell-to-nearest-neighbor distance) features. We analyzed the predictive value of implementing these biomarkers individually and then in an iterative sequential manner to distinguish tissue samples with CLL, aCLL, and RT. In some embodiments, a model based on these four morphologic biomarker attributes was able to achieve a robust analytic accuracy. These identified biomarkers can be used to assist in the diagnostic evaluation of tissue samples from subjects with CLL who develop aggressive disease features. In some embodiments, using a heat value based on nuclear size and nuclear intensity allowed for visualization or identification of proliferation centers (PCs) and also could be used to diagnose tissue samples from subjects with CLL. Methods and systems using the biomarkers may accurately and efficiently diagnose levels of a disorder. Diagnosis may be performed with less biological sample (e.g., from a core-needle biopsy), resulting in a more rapid and minimally invasive biopsy.

Embodiments include a method for analyzing a plurality of lymphoid cells. The method may include receiving data of an image captured of the plurality of lymphoid cells in a biological sample from a subject. The data may include pixels. The method may also include identifying, in the data, first pixels corresponding to nuclei of the plurality of lymphoid cells. The method may further include measuring a value for nuclear size, a value for nuclear intensity, and a value for cellular density using the first pixels. In addition, the method may include determining, using the measured values, a classification of leukemia or lymphoma in the subject.

Embodiments may include a method for analyzing a plurality of lymphoid cells. The method may include receiving data of an image captured of the plurality of lymphoid cells in a biological sample from a subject. The data may include pixels. The method may also include identifying, in the data, first pixels corresponding to the plurality of lymphoid cells. The method may further include segmenting, using the first pixels, each lymphoid cell of the plurality of lymphoid cells into second pixels corresponding to a first portion with a nucleus and third pixels corresponding to a second portion without a nucleus. Additionally, the method may include filtering, using the second pixels and the third pixels, the plurality of lymphoid cells to remove lymphoid cells having overlapping first portions to produce fourth pixels corresponding to a filtered plurality of lymphoid cells. The method may also include measuring a value for nuclear size, a value for nuclear intensity, and a value for cellular density using the fourth pixels corresponding to the filtered plurality of lymphoid cells. Furthermore, the method may include determining, using the measured values, a classification of leukemia or lymphoma in the subject.

Embodiments may include a method for analyzing a plurality of lymphoid cells. The method may include receiving data of an image captured of the plurality of lymphoid cells in a biological sample from a subject. The data may include pixels. The method may also include identifying, in the data, first pixels corresponding to the plurality of lymphoid cells. The method may further include segmenting, using the first pixels, each lymphoid cell of the plurality of lymphoid cells into second pixels corresponding to a first portion with a nucleus and third pixels corresponding to a second portion without a nucleus. In addition, the method may include filtering, using the second pixels and the third pixels, the plurality of lymphoid cells to remove lymphoid cells having overlapping first portions to produce fourth pixels corresponding to a filtered plurality of lymphoid cells. The method may also include measuring a value for nuclear size and a value for nuclear intensity for each lymphoid cell of the filtered plurality of lymphoid cells using the fourth pixels. The method may further include determining a value of a parameter using the measured values. In addition, the method may include comparing the value of the parameter to a reference value. The method may further include determining, using the comparison, a classification of leukemia or lymphoma in the subject.

Embodiments may include a method for analyzing a plurality of lymphoid cells. The method may include receiving data of an image captured of the plurality of lymphoid cells in a biological sample from a subject. The data may include a plurality of pixels. The method may also include identifying, in the data, first pixels corresponding to the plurality of lymphoid cells, the plurality of pixels comprising the first pixels. The method may further include segmenting, using the first pixels, each lymphoid cell of the plurality of lymphoid cells into second pixels corresponding to a first portion with a nucleus and third pixels corresponding to a second portion without a nucleus. Additionally, the method may include filtering, using the second pixels and the third pixels, the plurality of lymphoid cells to remove lymphoid cells having overlapping first portions to produce fourth pixels corresponding to a filtered plurality of lymphoid cells. The method may also include measuring a value for nuclear size and a value for nuclear intensity for each lymphoid cell of the filtered plurality of lymphoid cells using the fourth pixels. The method may include dividing the data of the image into a plurality of tiles. For each tile of the plurality of tiles, a value of a parameter may be determined using the measured values of nuclear size and the measured values of nuclear intensity for lymphoid cells corresponding to the fourth pixels in the tile. The method may further include identifying, using the values of the parameter, a subset of the plurality of tiles as representing cells with increased mitotic activity.

Embodiments may include a method for analyzing a plurality of lymphoid cells. The method may include receiving data of an image captured of the plurality of lymphoid cells in a biological sample from a subject. The data may include a plurality of pixels. The method may also include identifying, in the data, first pixels corresponding to the plurality of lymphoid cells, the plurality of pixels comprising the first pixels. The method may further include segmenting, using the first pixels, each lymphoid cell of the plurality of lymphoid cells into second pixels corresponding to a first portion with a nucleus and third pixels corresponding to a second portion without a nucleus. Additionally, the method may include filtering, using the second pixels and the third pixels, the plurality of lymphoid cells to remove lymphoid cells having overlapping first portions to produce fourth pixels corresponding to a filtered plurality of lymphoid cells. The method may also include measuring a value for nuclear size and a value for nuclear intensity for each lymphoid cell of the filtered plurality of lymphoid cells using the fourth pixels. The method may include dividing the data of the image into a plurality of tiles. For each tile of the plurality of tiles, a value of a parameter may be determined using the measured values of nuclear size and the measured values of nuclear intensity for lymphoid cells corresponding to the fourth pixels in the tile. The method may further include determining a classification of leukemia or lymphoma using the values of the parameters for the plurality of tiles.

Embodiments include methods for determining leukemia or lymphoma levels using lymphoid images. Embodiments use unsupervised clustering to divide lymphoid cells into different cell types. The feature values of properties of these cell types are determined. A classification of a level of leukemia or lymphoma can be determined from the feature values of the properties of the cell types. Embodiments may include using six or fewer feature values. Despite the small number of feature values, classification of the level of leukemia or lymphoma can be determined with high accuracy, including high sensitivity and specificity.

In embodiments, methods may include receiving data of an image captured of the plurality of lymphoid cells in a biological sample from a subject. The data may include pixels. In addition, methods may include identifying, in the data, first pixels corresponding to nuclei of a set of the plurality of lymphoid cells. The set may satisfy one or more criteria. Methods may also include for each cell of the set of the plurality of lymphoid cells, measuring a value for nuclear size and a value for nuclear intensity using the first pixels. Methods may further include classifying each lymphoid cell of the set of the plurality of lymphoid cells into one of a plurality of cell types using the value for nuclear size of the cell and the value for nuclear intensity of the cell. Methods may include determining statistical feature values for one or more properties of the cells of the cell types. Methods may also include determining, using the statistical feature values for the one or more properties of each of the plurality of cell types, a classification of leukemia or lymphoma in the subject.

Aspects also include a computer product comprising a non-transitory computer readable medium storing a plurality of instructions that when executed control a computer system to perform methods described herein. Aspects also include a system comprising means for performing methods described herein. Further aspects include a system including one or more processors configured to perform any methods described herein. Additional aspects include a system including modules that respectively perform the steps of any methods described herein.

A better understanding of the nature and advantages of embodiments of the present invention may be gained with reference to the following detailed description and the accompanying drawings.

### BRIEF DESCRIPTION OF THE DRAWINGS

FIGS. 1A-1G show an overview of the study design for analyzing lymphoid images according to embodiments of the present invention.

FIG. 2 is a table of subject demographic features and slide characteristics used for determining biomarkers according to embodiments of the present invention.

FIGS. 3A and 3B illustrate overlapping nuclei and nuclear segmentation according to embodiments of the present invention.

FIGS. 4A-4D show graphs analyzing nuclear size, nuclear intensity, cellular density, and cellular distance according to embodiments of the present invention.

FIGS. 5A-5G show results of univariate testing diagnostic predictions as confusion matrices according to embodiments of the present invention.

FIG. 6 shows the performance of the repeated splitting analysis and using different sets of biomarkers according to embodiments of the present invention.

FIG. 7 is a flowchart of an example process associated with methods for determining leukemia or lymphoma levels using lymphoid images according to embodiments of the present invention.

FIGS. 8A, 8B, and 8C show going from an image of a region of interest to heat map with tiles of heat values according to embodiments of the present invention.

FIG. 9 shows the pipeline from an image of a region of interest to generating a heatmap, proliferation centers, and heat value histograms according to embodiments of the present invention.

FIG. 10 shows boxplots of the mean heat values for regions of interest for different lymphoma according to embodiments of the present invention.

FIG. 11 is a flowchart of a method for analyzing a plurality of lymphoid cells and identifying proliferation centers according to embodiments of the present invention.

FIG. 12 is a flowchart of a method for analyzing a plurality of lymphoid cells to determine leukemia or lymphoma levels according to embodiments of the present invention.

FIG. 13 is a flowchart of a method for determining leukemia or lymphoma levels using heat values in lymphoid images according to embodiments of the present invention.

FIG. 14 shows a table of the characteristics of the subjects according to embodiments of the present invention.

FIGS. 15A-15E show an overview of the study design for analyzing lymphoid images according to embodiments of the present invention.

FIG. 16 is a table showing the list of three different sets of features used to classify the tissue sample according to embodiments of the present invention.

FIG. 17 shows three different ways of analyzing a tissue sample according to embodiments of the present invention.

FIG. 18 illustrates the procedures of using convolutional neural network (CNN) to extract region of interest (ROI) features according to embodiments of the present invention.

FIGS. 19A and 19B show graphs of the different cell subtypes according to embodiments of the present invention.

FIGS. 20A-20F show accuracy and AUC results of analyzing disease progression using features generated from different techniques according to embodiments of the present invention.

FIGS. 21A and 21B show correlation and importance of features according to embodiments of the present invention.

FIGS. 22A and 23B show results of unsupervised clustering stability analysis and from repeated splitting analysis according to embodiments of the present invention.

FIG. 23 is a table showing identities of four features used to classify a disorder according to embodiments of the present invention.

FIG. 24 is a flowchart of an example process for analyzing a plurality of lymphoid cells according to embodiments of the present invention.

FIG. 25 illustrates a measurement system according to embodiments of the present invention.

FIG. 26 illustrates a computer system according to embodiments of the present invention.

### DETAILED DESCRIPTION

Machine learning algorithms on digital pathology images can improve diagnostic accuracy and can correlate with biologic subsets. Limited studies have evaluated deep learning algorithms to evaluate lymphoid proliferations, and their main focus has been to distinguish benign from malignant conditions and different subtypes of lymphoma. Most studies have adopted a patch-wise strategy for whole-slide image analysis, which entails making diagnostic predictions for patches using a convolutional neural network (CNN) followed by fusing patch predictions to render a final slide diagnosis. A limitation of the deep learning model is the lack of clinical relatability, as using CNNs derives uninterpretable features from patches without a systematic stepwise approach of disease-specific feature identification.

To test our hypothesis and develop our models, we used chronic lymphocytic leukemia (CLL) as a disease prototype, as it is among the most common lymphoid neoplasms in adults. Chronic lymphocytic leukemia/small lymphocytic lymphoma (CLL) is one of the most prevailing adult leukemia in western countries and among the most common lymphoid neoplasms in adults. In addition, CLL is known to undergo stepwise progression to aggressive disease. Typical CLL is comprised of monomorphous sheets of small, mature lymphocytes interspersed by areas termed “proliferation centers” containing larger cells that include prolymphocytes and large cells referred to as paraimmunoblasts. Whereas CLL is generally an indolent disease, approximately 10% of CLL patients undergo histologic transformation to aggressive lymphomas, namely diffuse large B-cell lymphoma (DLBCL), also known as Richter's transformation (RT). An intermediate stage of progression, termed accelerated phase CLL (aCLL), demonstrates overlapping clinical and morphologic features of both CLL and RT. The morphologic assessment of CLL, aCLL, and RT can be diagnostically challenging, particularly in small needle core biopsy specimens. We thought this stepwise progression model of CLL, aCLL, and RT would be a promising substrate to test AI models. Providing an accurate diagnosis in patients with the evolving disease is of paramount importance, as a diagnostic upgrade to a more aggressive disease results in a management rise to more intensive therapeutic options.

### I. Lymphoid Image Biomarkers

Artificial intelligence (AI) models to evaluate lymphocytic proliferations remain limited. Meanwhile, several persistent and clinically-relevant areas of diagnostic overlap in the evaluation of lymphomas in tissues can benefit from machine-assisted diagnostic evaluation. We hypothesized that generating patterns integrating cell morphologic traits (e.g., nuclear size and nuclear intensity) and spatial patterns (e.g., cellular density and cell-to-nearest-neighbor distance) would be uniquely suited for enhancing diagnostic accuracy of lymphoid neoplasms. We demonstrate herein that the sequential implementation of key morphologic biomarkers (e.g., nuclear size, nuclear intensity, cellular density, and/or cell-to-nearest-neighbor distance) improved the accuracy of assessing CLL, aCLL, and RT.

Embodiments of the present invention by using biomarkers are able to accurately and efficiently assess a level of a disease or a progression of a disease. Surprisingly, only three or four biomarkers can be used to generate accurate results. Using only three or four biomarkers permits computational efficiency. Additionally, methods and systems described herein can use small samples, including core needle biopsy samples. Moreover, accurate analysis is achievable through using a small region of a whole slide image rather than most or all of the whole slide image. Assessing the progression of the disease can be accurately performed, even when assessing progression of a disease may be more challenging than simply identifying the existence of a disease.

### A. Lymphoid Images Study

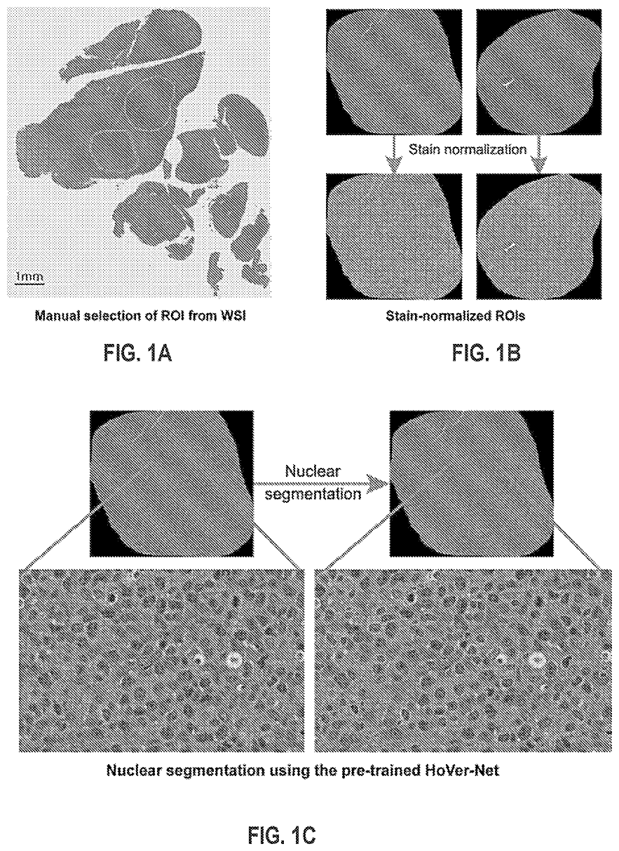

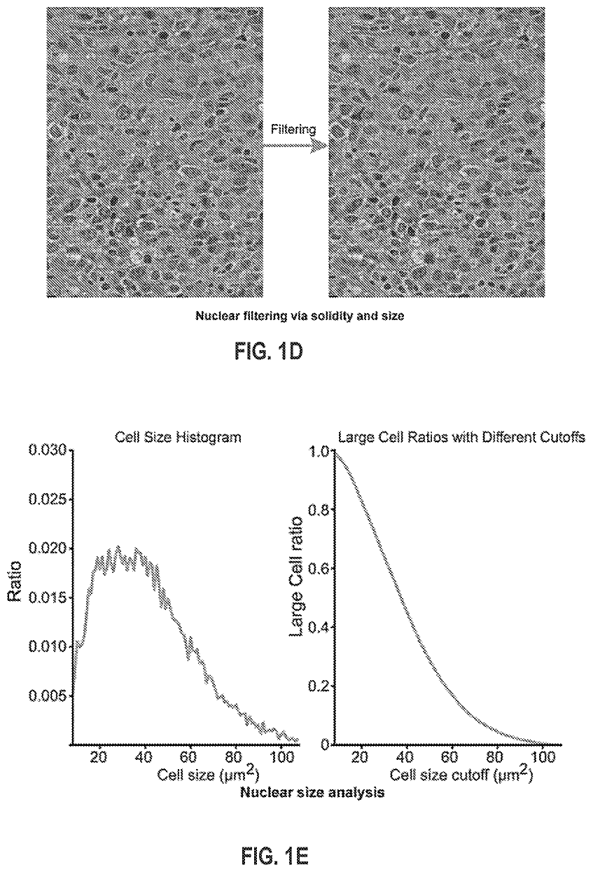

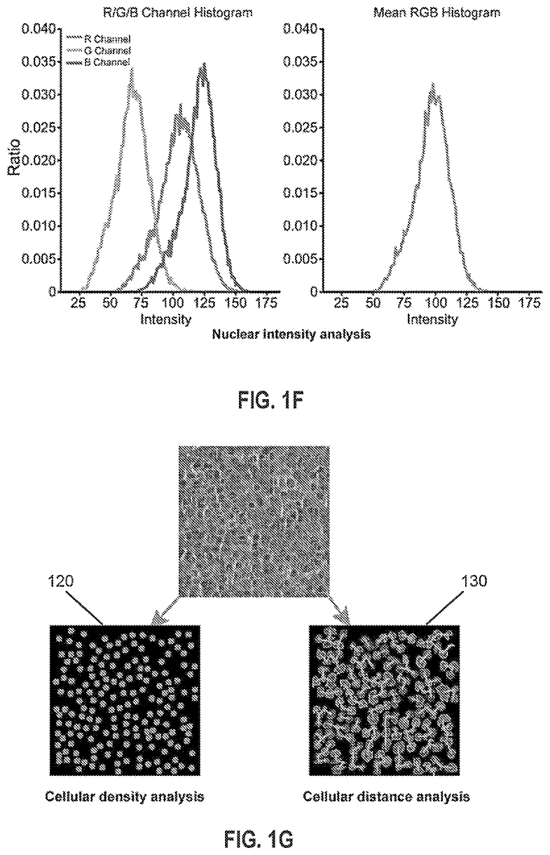

FIGS. 1A-1G show an overview of the study design for analyzing lymphoid images. In brief, we collected and analyzed digital slides of subjects diagnosed with at least one of the following entities: CLL, aCLL and/or RT. The figures illustrate a pipeline from selecting regions of interest to measuring values of biomarkers. FIG. 1A shows starting with digitized whole-slide images (WSI) and manually selecting regions of interest (ROIs). FIG. 1B shows applying stain normalization in the regions of interest. FIG. 1C shows nuclear segmentation to delineate malignant nuclei after stain normalization. FIG. 1D shows using nuclear filtering of solidity and size to exclude overlapping nuclei. FIG. 1E shows characterizing each ROI through quantification of nuclear size. FIG. 1F shows characterizing each ROI through quantification of nuclear intensity based on nuclear color composition. FIG. 1G shows image 120 illustrating determination of cellular density and image 130 illustrating determination of cell-to-nearest-neighbor distance.

We analyzed the predictive value of implementing these “biomarkers” individually (nuclear size, nuclear intensity, cellular density, and cell-to-nearest-neighbor distance). We also assessed the synergistic effects of sequentially adding these biomarkers to enhance diagnostic accuracy.

### 1. Materials and Methods

Materials and methods involved analyzing images from histology slides. The images were then analyzed for different features. These features were then used in a model to diagnose diseases in subjects.

### a) Subject Groups and Histology Slides

We retrospectively searched subjects with hematolymphoid diseases clinically evaluated at The University of Texas MD Anderson Cancer Center (UTMDACC) between Feb. 1, 2009 and Jul. 31, 2021. In total, 125 subject biopsy specimens were eligible consisting of 69 CLL slides (from 44 subjects), 44 aCLL slides (from 34 subjects), and 80 RT-DLBCL slides (from 47 subjects). Inclusion in this study was based on the following criteria: 1) availability of archived glass slides and digital slides; 2) lymph node biopsy confirmed diagnosis; 3) availability of clinical and laboratory data to support the diagnosis in the electronic medical records. Microscopic diagnosis was confirmed on all cases by two hematopathologists, and challenging cases were resolved by a third hematopathologist. CLL and RT were defined as described by World Health Organization Classification (Swerdlow S H C E, Harris N L, Jaffe E S, Pileri S A, Stein H, Thiele J: WHO Classification of Tumours of Haematopoietic and Lymphoid Tissues. Revised 4th Edition ed. Lyon, France: IARC, 2017), and aCLL was defined as described previously (Gine E, Martinez A, Villamor N, Lopez-Guillermo A, Camos M, Martinez D, Esteve J, Calvo X, Muntanola A, Abrisqueta P, Rozman M, Rozman C, Bosch F, Campo E, Montserrat E: Expanded and highly active proliferation centers identify a histological subtype of chronic lymphocytic leukemia (“accelerated” chronic lymphocytic leukemia) with aggressive clinical behavior. Haematologica 2010, 95:1526-33). A sizable subset (45.5%) of histology slides represented referred material from other institutions, increasing the diversity of slide sources.

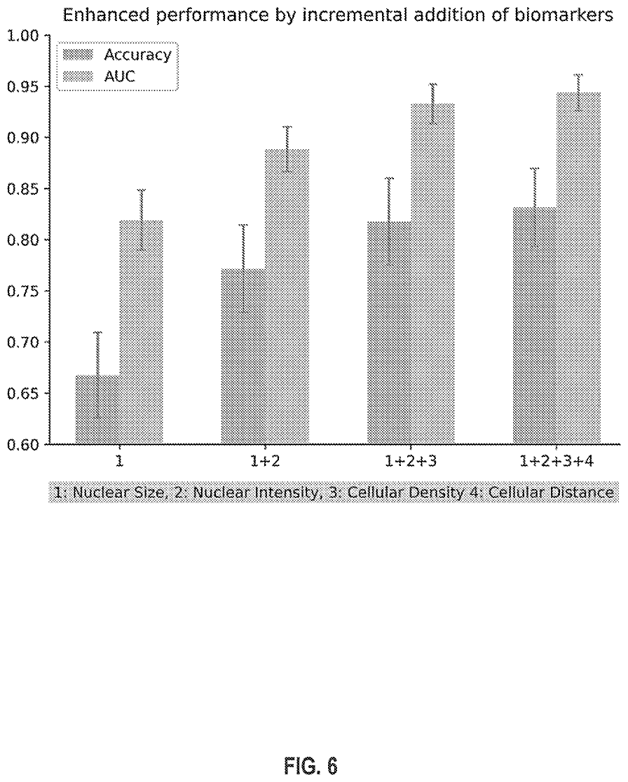

Glass slides stained with hematoxylin and eosin (H&E) were scanned using an Aperio AT2 scanner at ×20 (0.50 μm/pixel). Scanning was performed in 3 batches within the same day, using the same scanner. The overall study group was divided equally into separate training and testing cohorts. To mitigate random effects and ensure balanced splitting, we stratified subjects by matching age (above or below 60 years), gender, overall survival (>40 or <40 months for CLL, >19 or <19 for aCLL, >9 or <9 months for RT), case source (in house vs referred consultation cases), and biopsy techniques (core-needle vs excisional). Details of subject demographic features and slide characteristics as well as the splitting of training and testing are outlined in FIG. 6.

FIG. 6 is a table 600 showing the number of subjects (e.g., patients) corresponding to each disease classification for different subject demographic features and slide characteristics. The first ten columns correspond to the training set. The second ten columns correspond to the testing set. The training set and testing set include the same ten categories for the columns. Age refers to the age in years at the time of the CLL diagnosis. Gender is divided into male (“M”) and female (“F”). The case source includes an in-house (“I”) and outside institution (“O”) cases. In-house cases are cases in the MD Anderson Cancer Center. Biopsy technique is divided into excisional biopsy (“E”) and core needle biopsy (“CNB”). OS refers to the overall survival. Overall survival refers to the time interval in months between the date of diagnosis and the date of death. The inequality in the table indicates if the patent's overall survival is longer or shorter than the number. For CLL subjects, the time interval is 40 months; for aCLL subjects, the time interval is 19 months; and for RT subjects, the time interval is 9 months.

### b) Image Analysis

Images were analyzed by selecting regions of interest. Nuclear segmentation in the regions of interest is performed. Features, such as nuclear size, nuclear intensity, cellular density, and/or cell-to-nearest neighbor distance, may be determined using results from nuclear segmentation.

### (1) Selection of Regions of Interest (ROI)

A regular lymph node excisional biopsy specimen digital slide (e.g., 80,000×50,000 pixels in the level 0 of the digital slide pyramidal storage) contains more than two million cells. Analyzing a whole slide image is computationally expensive, and the quality of some regions of the digital slide in some instances may not be suitable for diagnosis. For example, several kinds of tissue processing artifacts including, tissue folding, crushing artifact, and tissue section thickness may result in a suboptimal whole-slide tissue assessment. To overcome these pre-analytic impediments, we adopted the region of interest (ROI) approach for cell morphology analysis. Diagnostically informative ROIs with the highest tissue quality were manually selected for subsequent analysis (e.g., FIG. 1A). The ROI were selected in a manner that is representative of the final diagnosis. For example, if a case was finally diagnosed as RT, the ROI was selected to include the predominant population on the whole slide, the large cells, and not the small residual CLL foci in the background.

The number of ROIs per slide averaged 2.4 and ranged from 1 to 8. The number of ROIs in CLL, aCLL, and RT were 159, 141, and 165, respectively. To ensure the availability of sufficient cells in each ROI, we limited the minimum width and height of all selected ROIs to 500 pixels, which corresponded to 0.25 mm. Total sizes of ROIs were overall very similar. Stain normalization was performed on all ROIs prior to further processing (e.g., FIG. 1B).

### (2) Nuclear Segmentation

Nuclear segmentation is performed before carrying out cellular feature analysis. As illustrated in FIG. 1C, we performed nuclear segmentation of selected ROIs using the robust and highly generalized Hover-Net (Graham S V Q, Raza S E A, Azam A, Tsang Y W, Kwak J T, Rajpoot N: Hover-Net: Simultaneous segmentation and classification of nuclei in multi-tissue histology images. Med. Image Anal. 2019, 58:101563). We avoided time-consuming cell annotation and fine-tuning processes by directly applying the Hover-Net model, which is pre-trained on the PanNuke dataset. The PanNuke dataset covers exhaustive nuclear labels across 19 different tissue types with H&E staining from The Cancer Genome Atlas (TCGA) (G J: PanNuke: An Open Pan-Cancer Histology Dataset for Nuclei Instance Segmentation and Classification. European Congress on Digital Pathology 2019). The diversity of tissue types in the PanNuke dataset enables adequate generalization of the pre-trained Hover-Net model. The nuclear segmentation results were visually checked by a hemapathologist to confirm their quality.

### (3) Nuclear Filtering

The Hover-Net leverages the encoded information within the vertical and horizontal directions to tackle cell-clustering issues and thus provides high nuclear segmentation performance. Yet, overlapping nuclei still pose substantial challenges to nuclear segmentation, as many nuclei can merge together.

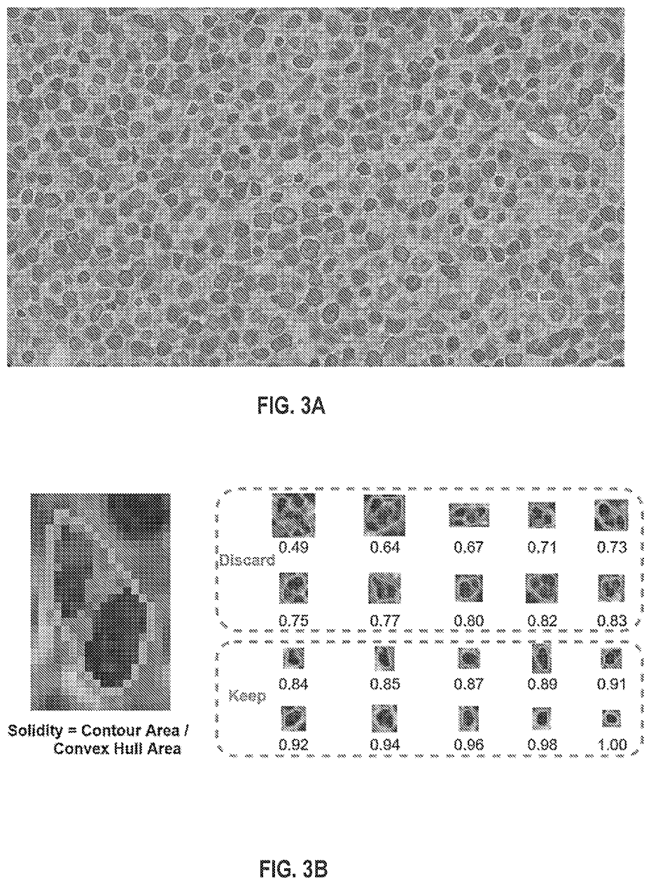

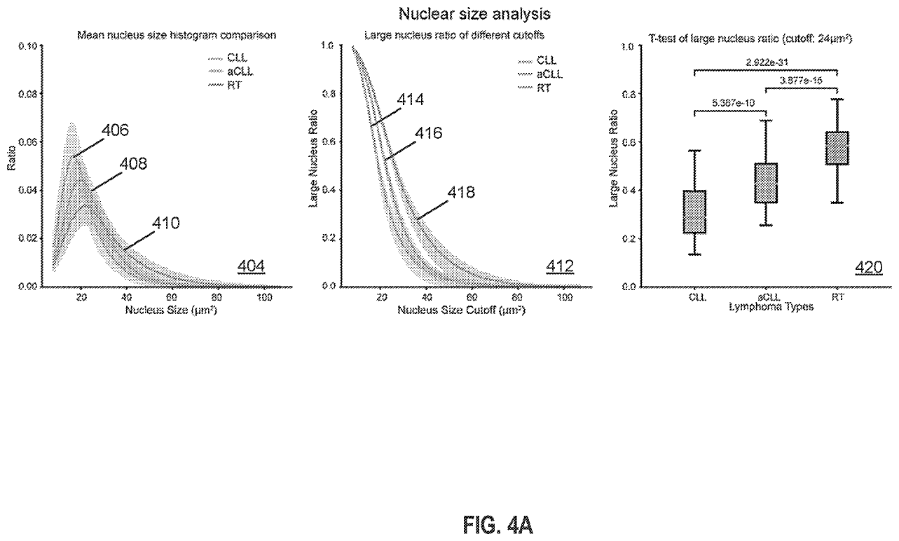

FIG. 3A shows arrows pointing to areas of the image with overlapping nuclei. The overlapping nuclei may affect the sensitivity of nuclear size analysis. Overlapping nuclei may give the false impression of large cell nuclei. Overlapping nuclei result in concavities in the image. To overcome this challenge, we used the solidity feature to filter out segmented cells with overlapping nuclei by considering the concave contour shape of two overlapping nuclei (e.g., shapes indicated by red arrows in FIG. 3A and FIG. 1D).

FIG. 3B shows the solidity calculation technique and illustrates nuclear segmentation samples. The solidity value is defined as the ratio of segmented nuclear contour area (red area) to its convex hull area (green area). We only kept segmented nuclei with a solidity value larger than 0.84 (we noticed that by applying this threshold, at least 98% of nuclei were retained, while overlapping nuclei were largely removed). In addition, we focused our attention on segmented nuclei with pixel values between 32 and 432, corresponding to 8 μm² and 108 μm², as this approach represents a known biologic nuclear size range on histologic slides. Nuclei not satisfying solidity or size requirements may be discarded and not analyzed further.

We performed a quantitative evaluation for the Hover-Net segmented cells on 15 manually annotated patches of size 256*256, including 5 CLL, 5 aCLL, and 5 RT. Since we had a cell filtering process, we evaluate the Dice score (a tool for evaluation of cell segmentation via AI-based algorithms) for those remaining cells after the filtering process. For each individual segmented cell, we found its corresponding cell from pathologists' manual annotations, and calculated the Dice score. The mean Dice scores of CLL, aCLL and RT were 0.824, 0.835, and 0.817, respectively. The overall mean Dice score was 0.825 in the 15 total evaluated cases. The Dice scores Hover-Net reported on Kumar, CoNSeP, and CPM-17 datasets are 0.826, 0.853, and 0.869, respectively. Cell segmentation performance on our dataset is compatible to Hover-Net's reported performance, demonstrating the generalization capacity of pre-trained Hover-Net on the PanNuke dataset. We believe that cell segmentation with a Dice score over 0.8 is sufficient for downstream cellular feature extraction. Consequently, after acquiring a reasonable nuclear segmentation result for each ROI, we set the stage for nuclear size analysis.

### (4) Nuclear Size

Neoplastic cells in CLL characteristically have scant cytoplasm, and thus cell size is mainly the reflection of nuclear size. Neoplastic cells in RT, on the other hand, have more cytoplasm in comparison to CLL, but retain a high nuclear to cytoplasmic ratio rendering cell size the reflection of mainly nuclear size, similar to CLL. The cellular features in cases of aCLL fall between morphologic attributes of CLL and RT. Using this rationale, for each ROI, we generated a nuclear size histogram based on nuclear segmentation results (e.g., FIG. 1E). By setting a large size cutoff, nuclei were partitioned into two categories: small and large. The large nuclear ratio, defined as the number of large nuclei over the total number of nuclei, was computed for each cutoff. We first sought to identify a cutoff value to separate the three groups (CLL, aCLL, and RT). We assigned the average large nuclear ratios of CLL, aCLL, and RT as R_{CLL}, R_{acLL}, and R_{RT}, and then defined (R_{RT}−R_{aCLL})*(R_{acLL}−R_{CLL})*(R_{RT}−R_{CLL}) as the objective function is to find the best cutoff value. By maximizing the objective function on training ROIs, we discovered that the value 24 μm was the nuclear size cutoff that best distinguished CLL from aCLL and RT with our data set.

### (5) Nuclear Intensity

To determine nuclear intensity defined by nuclear color composition, we obtained individual nuclear images by overlapping nuclear segmentation analysis and corresponding ROI images. The cellular intensity of CLL cells is mainly a reflection of nuclear intensity. Neoplastic cells in RT on the other hand, although they have a moderate amount of cytoplasm in comparison to CLL, demonstrate a high nuclear to cytoplasmic ratio also rendering cellular intensity the reflection of mainly nuclear intensity. Cellular features of aCLL cells fall in between CLL and RT morphologic attributes. Using this rationale for each nucleus, we first measured the mean intensity of its R (red), G (green), and B (blue) channels. We then computed the histograms of R, G, and B intensities for each ROI, calculated the mean R, G, and B intensities and obtained an overall mean RGB intensity for each ROI (e.g., FIG. 1F).

### (6) Cellular Density

Beside individual nuclear properties (e.g., size and intensity), we explored cellular density (e.g., number of cells per ROI) as a potential additional biomarker to refine diagnostic accuracy (e.g., using analysis with image 120 in FIG. 1G). For each ROI, we counted the number of cells after nuclear segmentation and filtering, and we assigned the cell number to N_{cell}. In addition, we obtained the physical size of each ROI, Sᵣₒᵢ, by counting pixels it contains and multiplying slide digitization magnification (0.50 μm/pixel). We defined the cellular density as N_{cell}/Sᵣₒᵢ.

### (7) Cell-to-Nearest-Neighbor Distance

We then sought to investigate the distance between neighboring cells (e.g., see analysis with image 130 in FIG. 1G). For each cell inside a given ROI, we measured its Euclidean distances to other cells via their spatial coordinates. We identified the cell with the smallest Euclidean distance as the nearest neighbor, and its distance to the cell of interest was considered as the cell-to-nearest-neighbor distance. We then calculated the mean value of all cell-to-nearest-neighbor distances in a given ROI.

### c) Disease Diagnosis Model

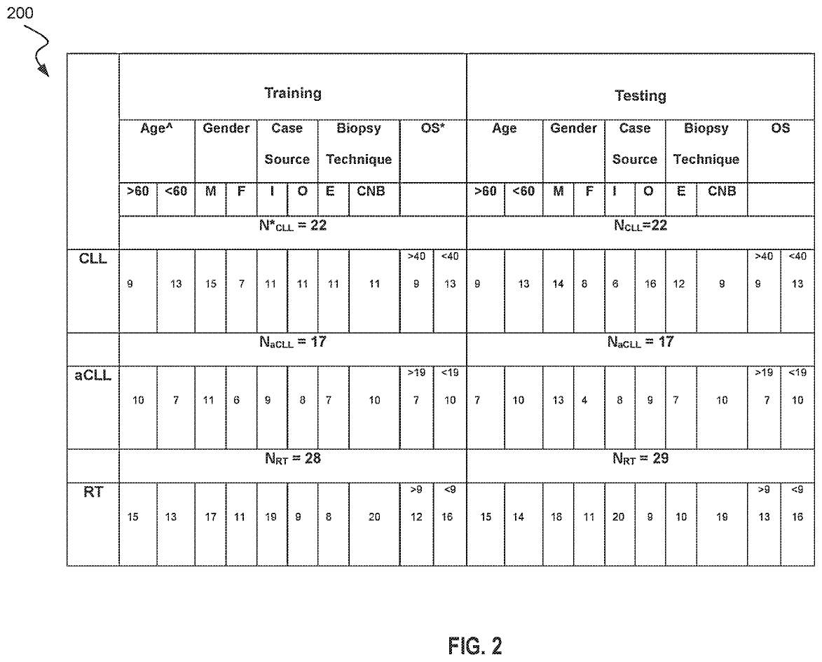

Based on the above cellular and architectural biomarkers (nuclear size, nuclear intensity, cellular density, and cellular distance), we conducted two diagnostic experiments on annotated ROIs from the three groups. In the first experiment, we trained the disease diagnosis model using the training subject cohort and evaluated the performance of the model using the testing subject cohort. Details about the training and testing cohorts are outlined in FIG. 2.

We aimed to balance the training and validation datasets by controlling variables such as age, gender, case source, biopsy techniques, and overall survival. In the second diagnostic experiment, we performed repeated splitting analysis in which we combined the training and testing cohorts and then randomly split them into training and testing sets based on subjects with a ratio of 1:1 and repeating 100 times. Subject-based splitting was performed to avoid running into ROIs belonging to the same subjects in both the training and testing sets, which may lead to information leakage. We then reported the mean and standard deviation of disease diagnosis on the 100-times splitting basis to avoid any potential biases that might be caused by one-time splitting, and we further validated our model's generalizability and robustness. The repeated splitting test aims to strengthen the statistical significance of the analysis. In fact, when conducting a t-test, a sample size n≥30 is treated as large based on the statistical theory. We chose to repeat splitting 100 times in our model as a balance between sample size and computational cost. We conducted experiments to explore the effects of repeated splitting times. We experimented with repeated splitting times ranging from 60 to 200 and found that the diagnostic performance is quite consistent among different numbers within this range. For both diagnostic experiments, we used the Random Forest classifier from python scikit-learn package (version 0.24).

### d) Statistical Analyses

Biomarker statistical significance analysis of our diagnostic model was calculated by applying Welch's t-test, using python SciPy package (version 1.6.1). The receiver operating characteristics (ROC) curve analysis and area under the curve (AUC) were used to assess the prediction capability of the proposed imaging models.

### 2. Results

Nuclear size, nuclear intensity, cellular density, and/or cell to nearest-neighbor distance were used as biomarkers. These biomarkers showed good performance in diagnosing disease.

### a) Individual Biomarker Analysis

Nuclear size, nuclear intensity, cellular density, and cell to nearest-neighbor distance are analyzed for relationships to different lymphoma types.

### (1) Nuclear Size

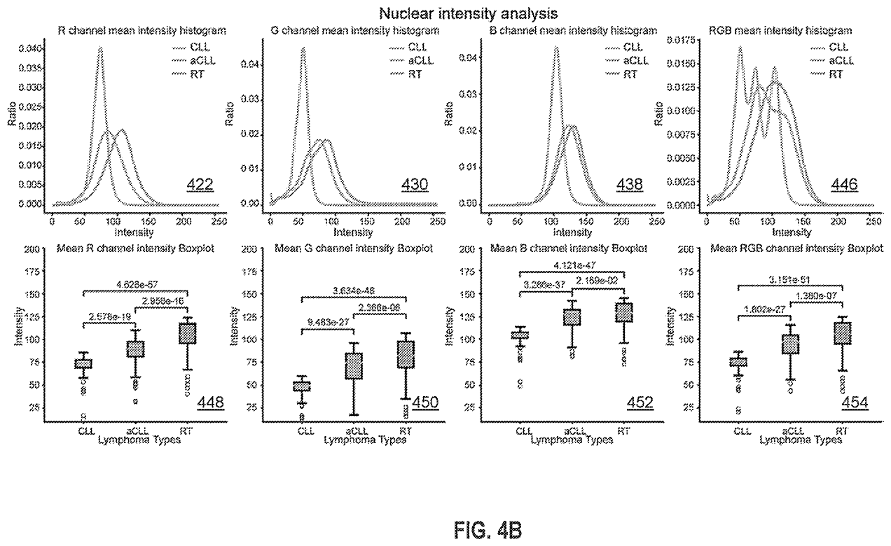

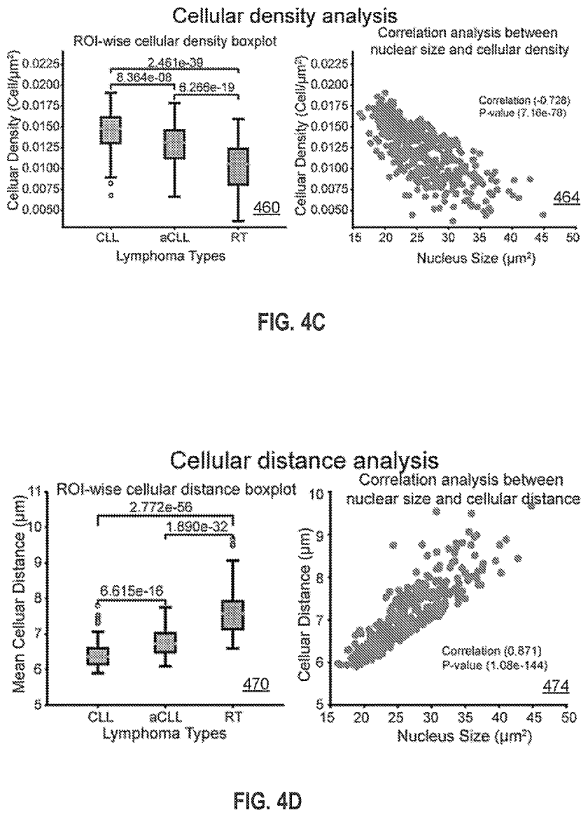

FIG. 4A shows graphs for analyzing nuclear sizes. For each ROI, we drew a nuclear size histogram (graph 404) and calculated the mean and standard deviation of the histograms for CLL (green line 406), aCLL (orange line 408), and RT (purple line 410). Nuclei in CLL ROIs clustered around 20 μm². In aCLL cases, ROIs had a mean size close to 20 μm² but a sizeable portion of ROIs harbored cells with larger nuclear size, resulting in a more widely spread nuclear size histogram in comparison with CLL cases. In contrast, RT ROIs harbored a higher number of cells with nuclear size larger than 20 μm², resulting in a mean histogram extruding on regions with nuclear size larger than 20 μm².

Graph 412 shows the large nuclear ratios of ROIs belonging to CLL (green line 414), aCLL (orange line 416), and RT (purple line 418) with varying cutoffs. On a wide range of cutoff values, CLL and aCLL ROIs were found to have closer large nuclear ratios, whereas RT ROIs demonstrated larger nuclear ratios in comparison to CLL and aCLL. Using the proposed objective function (R_{RT}−R_{aCLL})*(R_{acLL}−R_{CLL})*(R_{RT}−R_{CLL}), we found that the cutoff value 24 μm² yielded the best separation among the three groups on training data. Based on this cutoff, we computed the large nuclear ratio for each ROI, as illustrated in the boxplot of graph 420. A t-test of large nuclear ratio, using a cutoff of 24 μm², demonstrates statistically significant differences among CLL, aCLL and RT ROIs. The large nuclear ratios exhibited a crescendo trend from CLL to aCLL and into RT with a statistically significant difference (p<0.05). However, based on this biomarker alone, there was still considerable overlap between CLL and aCLL, as well as aCLL and RT. To enhance the separation among the 3 groups, we sequentially added three more biomarkers, as outlined below.

### (2) Nuclear Intensity

FIG. 4B shows graphs for nuclear intensity analysis. We plotted the mean nuclear intensity histogram for the CLL, aCLL, and RT ROIs using the red (“R”) channel (graph 422), green (“G”) channel (graph 430), blue (“B”) channel (graph 438), and mean RGB channel (graph 446). The aCLL and RT ROIs showed higher mean values in different channels, whereas nuclei in the CLL ROIs demonstrated the smallest values overall. We then plotted the mean ROI nuclear intensity across the four different channels as box plots for the three groups in as illustrated in graphs 448, 450, 452, and 454. CLL ROIs had smaller mean levels across all channels compared with aCLL and RT (p<0.05). There were also significant differences between the mean ROI levels between aCLL and RT cases, (p<0.05) but the differences between aCLL and RT were less prominent compared to the differences between CLL and aCLL, as both mean ROI levels in B channel centered around the same value (˜130).

### (3) Cellular Density

To enhance diagnostic performance, we sought to use cellular density, which may be calculated as cell aggregates per square micrometer within an ROI. In FIG. 4C, we plotted cellular densities in CLL, aCLL, and RT ROIs in graph 460. We observed that the mean cellular density decreases significantly from CLL to RT ROIs. Although CLL and aCLL ROIs demonstrated closer cell density values when compared with RT, all three pairs (CLL/aCLL, aCLL/RT, CLL/RT) showed significant cellular density differences (p<0.05). We posited that the cellular density results in a given square micrometer of RT ROI is occupied by a smaller number of large cells, versus a larger number of smaller cells in a CLL or aCLL ROIs. To explore this point, we plotted the ROI mean cell size and cell density values in graph 464 and indeed found a negative correlation between cell size and cell density (Pearson correlation coefficient=−0.728, p=7.16e−78).

### (4) Cell to Nearest-Neighbor Distance

Cellular spatial relationships are not intuitively or easily employed by hematopathologists during manual morphologic evaluation. In view of the key implications that cellular composition and the tumor microenvironment play in the context of CLL disease evolution, we contended that extracting such data through a deep learning algorithm would add power to our diagnostic model. To explore the spatial relationships among cells, we investigated the cell-to-nearest-neighbor cell distance in CLL, aCLL, and RT ROIs.

FIG. 4D shows analysis of cellular distance. Graph 470 shows boxplots of the mean values of cellular distance for CLL, aCLL, and RT. RT ROIs showed the largest cell-to-nearest-neighbor distance among the three groups, whereas mean cellular distance is somewhat closer in aCLL and CLL ROIs; a significant difference (p<0.05) exists among any two of the three disease groups.

We posited for this parameter that a given square micrometer of RT ROI is occupied by a smaller number of large cells with resultant larger cellular distances, versus a larger number of smaller CLL and aCLL cells with smaller cellular distances in CLL or aCLL ROIs. To explore this point, we plotted the mean cell size and cell distance in graph 474 and indeed found a positive correlation between these two parameters (Pearson correlation coefficient=0.871, p=1.08e−144).

### b) Performance of the Disease Diagnosis Model

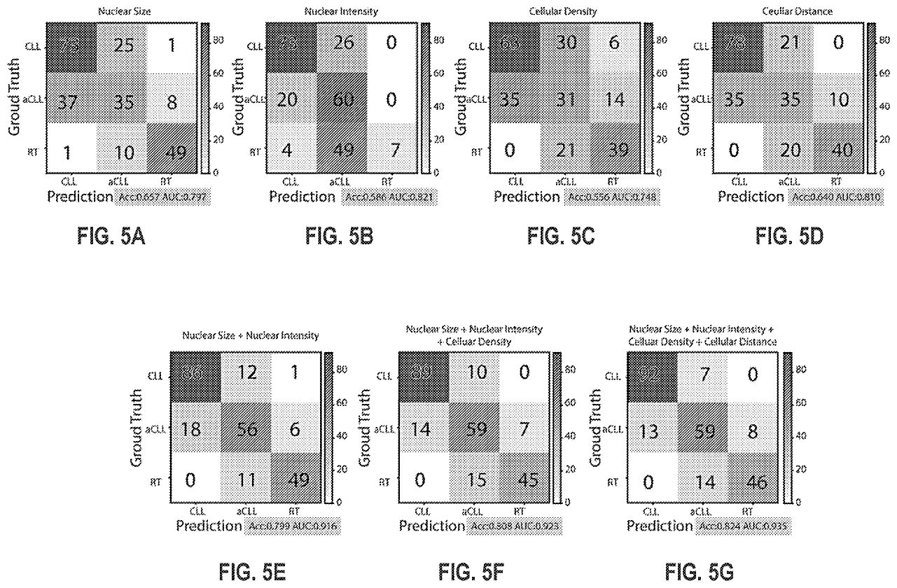

FIGS. 5A-5G show results of univariate testing diagnostic predictions as confusion matrices. The y-axis shows the ground truth stage of disease progression. The x-axis shows the predicted stage of disease progression. The number represents the number of samples corresponding to the ground truth and the prediction. The accuracy and the area under the curve are shown underneath each figure. Based on the univariate testing, the descending order of diagnostic predictive values of the biomarkers for accuracy was as follows: nuclear size (0.657) (FIG. 5A)>cellular distance (0.640) (FIG. 5D)>nuclear intensity (0.586) (FIG. 5B)>cellular density (0.556) (FIG. 5C). The descending order of diagnostic predictive values of the biomarkers for area under the curve (AUC) was as follows: nuclear intensity (0.820) (FIG. 5B)>cellular distance (0.810) (FIG. 5D)>nuclear size (0.797) (FIG. 5A)>cellular density (0.748) (FIG. 5C).

FIGS. 5E-5G show combining biomarkers to overcome overlapping morphologic attributes in the disease groups and enhance diagnostic accuracy. We started first by adding nuclear size and intensity, and both nuclear size and intensity demonstrated a higher accuracy and AUC compared to the individual biomarkers (FIG. 5E). FIG. 5F shows the addition of cellular density to nuclear size and nuclear intensity. FIG. 5G shows the addition of cellular distance to the other three biomarkers. By adding more biomarkers, we were able to increase the overall diagnostic performance as shown in the accuracy and AUC metrics. Namely, accuracy and AUC increased from 0.657 and 0.797, respectively, based solely upon cell size to 0.824 and 0.935, respectively, after nuclear intensity, cell density, and cell distance were added to nuclear size.

### c) Repeated Splitting Analysis

The above-described experiments were conducted in a single splitting round (one training and one testing set). To validate the stability and robustness of the four identified biomarkers, we combined all ROIs together and randomly split them into training and testing sets 100 times based on subject identification, and then evaluated diagnostic performance.

FIG. 6 shows the performance of the repeated splitting analysis. Similar to single training/testing splitting, a gradual performance enhancement is observed as more biomarkers are added, with a gradual increase in both the accuracy and AUC metrics. The statistical significance of both accuracy and AUC is enhanced as new biomarkers are added. When gradually adding three biomarkers, the p values for mean accuracy are 1.815e−41, 1.202e−12, and 0.015 between FIG. 6 groups 1 and 1+2, between groups 1+2 and 1+2+3, between groups 1+2+3 and 1+2+3+4, respectively. The p values for AUC are 9.064e−45, 7.045e−35, and 6.020e−05 between FIG. 6 groups 1 and 1+2, between groups 1+2 and 1+2+3, between groups 1+2+3 and 1+2+3+4, respectively. Furthermore, comparing the mean accuracy and AUC of the repeated splitting analysis with the accuracy and AUC from the first single training/test splitting shows that their values are close to each other when using the same number of biomarkers.

### 3. Discussion

However, such advances in applying deep learning algorithms to enhance diagnostic morphologic evaluation have been limited in the field of lymphoid neoplasia, where diagnostic evaluation is typically complex, as it relies on a broad constellation of morphologic attributes. In B-cell lymphomas, such attributes could be inherent to the neoplastic cells themselves and their ability to differentiate along a variety of pathways and, equally importantly, to the cellular and stromal components within the tumor microenvironment. These attributes are at play in biopsy samples from subjects with CLL, whose evaluation could occur within a variety of clinical contexts ranging from baseline incidental discovery of the disease to a suspicion of acceleration or transformation at any point during the course of the disease, often prompted by laboratory studies and/or exacerbation of systemic symptoms. Thus, while a diagnosis of CLL is usually straightforward, various factors contribute to making the diagnosis of aCLL and RT challenging, particularly in scant targeted needle core biopsy specimens in which the morphologic and architectural features may not be fully representative of the underlying disease.

Our design was inspired by clinical challenges encountered in clinical practice. In order to navigate and overcome inevitable diagnostic obstacles and impediments dictated by limited tissue samples and pre-analytical variables, we sought to conceptualize an AI-based disease diagnosis model to objectively assist in the evaluation of tissue samples from CLL subjects with clinical suspicion of disease progression. This clinically-oriented model is amenable to further optimization, validation, and possible implementation into pathology clinical practice, as the technology driving digital pathology unfolds and becomes more widely available. Adopting such AI-based models into clinical practice is predicted to improve diagnostic accuracy and reproducibility, and possibly identify biologic attributes and prognostic parameters.

The first building blocks of our diagnostic model included nuclear size and nuclear intensity. The number of larger nuclei is directly proportional to the extent of disease progression, and increased nuclear intensity is reflective of open and vesicular nuclear chromatin indicating increased nuclear activity, and translates into an increased cellular metabolism and proliferation rate. However, using these two aforementioned methods alone left persistent overlap across a number of CLL and aCLL ROIs. This overlap could be explained by the fact that aCLL is likely a mid-point in the spectrum of biologic progression of CLL, with more aggressive features that are not necessarily limited to nuclear size and intensity, but rather characterized by increased mitotic activity and expanded proliferation centers, among other features. In addition, a number of RT-DLBCL ROIs bled into the CLL cell size zone. This finding is likely explained by disease-biologic factors (e.g., fibrosis frequently occurs in RT, resulting in shrinking of otherwise large cells), and pre-analytical factors, including crush artifact and suboptimal tissue processing (e.g., thick tissue sections in RT result in decreased intensity), which affect tissue quality and architecture. These biologic and pre-analytical impediments triggered us to experiment with unconventional features such as cellular density and cell-to-nearest-neighbor distance in order to improve our disease diagnosis model.

We observed that a given CLL ROI, is populated by a large number of cells with small-sized nuclei, with decreased intercellular distance. In contrast, a given RT ROI is populated by a smaller number of cells with large-sized nuclei, with increased intercellular distance. By exploiting the cellular density and cell-to-nearest-neighbor distance, we enhanced the accuracy of our diagnostic model from 0.799 (based on combined nuclear size and intensity) to 0.808 with the addition of cellular density and 0.824 with all four biomarkers. Limitations to cell-to-nearest-neighbor distance analysis included, but were not restricted to, tissue fixation artifact and treatment-induced fibrosis, necrosis, and hemorrhage, creating an artifactual increase in cell distance. Tissue fixation artifacts are morphologic and/or architectural distortion caused during tissue processing, more specifically during formalin fixation step. Treatment-induced fibrosis, necrosis, and hemorrhage are all treatment-induced changes that could manifest in biopsied tissue post treatment. Fibrosis is increased collagen production by background fibroblasts; necrosis is cell death; and hemorrhage is red blood cell extravasation from vessels into tissue. Based on the results from the confusion matrix, aCLL remained the most challenging entity to fully characterize. However, an undeniable gradual improvement was observed as more biomarkers were added. In addition, our disease diagnosis model showed a remarkable performance in diagnosing CLL and RT-DLBCL, with minor overlaps of these two categories with aCLL, following the addition and analysis of all four biomarkers.

In contrast to other studies evaluating AI for the diagnosis of lymphoid neoplasms, we harnessed the power of deep learning to automate the key histologic features with clinical significance based on the hematopathology field rather than adopting a “black-box” scheme. Of note, our model with four clinically meaningful features has achieved an AUC of 0.935. A general challenge for deep learning models is their generalizability, rooted from model overfitting. In other words, these black-box models usually have a significant drop when testing on unseen datasets. By contrast, we expect our model to be more robust because it contains only four features to mitigate overfitting risk from a statistical perspective. Further, since these features are distilled from clinical practice, we anticipate that this model, with appropriate validation, is more likely to translated into clinical practice. Beyond tissue H&E slides, we envision our proposed analysis can also be transitioned into blood cell image analysis. Currently, three main classes of quantitative features are proposed to optimize morphology through blood cell digital image processing techniques to aid diagnosis of lymphomas, including geometric, color, and texture.

Our model performed robustly with 45.5% of analyzed histology slides being referred from other institutions. The hybrid design integrates deep learning with pathologist-annotated ROIs. Such annotation excludes artifacts and residual areas of clear CLL in cases of RT and aCLL, for example. We will be striving to develop an automated ROI selection algorithm in future iterations of our model.

In summary, our disease diagnosis model validates the assumption that designing new Allbiomarkers based on morphologic, architectural and microenvironmental features can boost diagnostic accuracy of disease entities or stages of progression within a single disease entity, as illustrated in this study assessing CLL, aCLL, and RT. The results of this study also highlight the importance of identifying more biomarkers in the future to enhance disease diagnosis performance in challenging clinical scenarios.

### 4. Example Methods

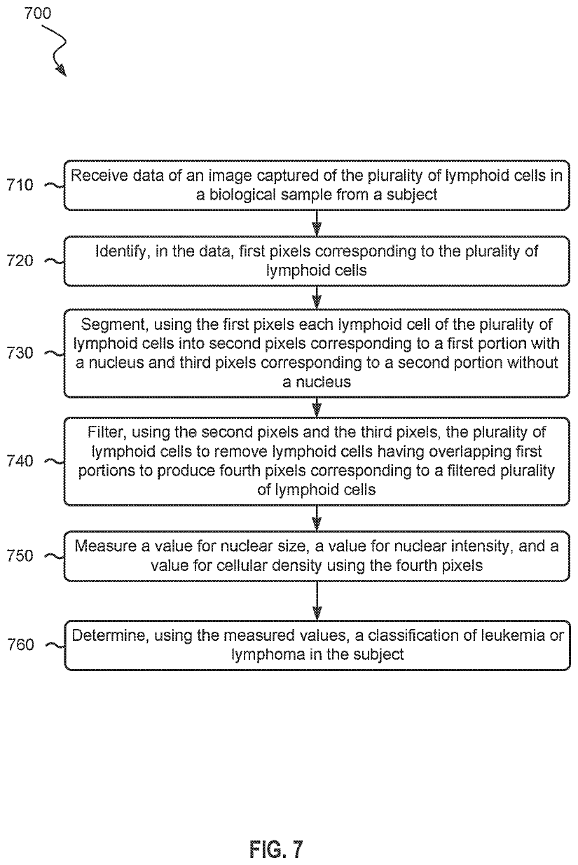

FIG. 7 is a flowchart of an example process 700 associated with methods for determining leukemia or lymphoma levels using lymphoid images. In some implementations, one or more process blocks of FIG. 7 may be performed by a system (e.g., system 2500), which may be or include a computer system. In some implementations, one or more process blocks of FIG. 7 may be performed by another device or a group of devices separate from or including the system. Additionally, or alternatively, one or more process blocks of FIG. 7 may be performed by one or more components of system 2500, such as imaging device 2510, detector 2520, logic system 2530, processor 2550, memory 2535, external memory 2540, storage device 2545, and/or treatment device 2560.

At block 710, data of an image captured of the plurality of lymphoid cells in a biological sample from a subject is received. The lymphoid cells may be treated with a stain. The stain may include hematoxylin and eosin. The plurality of lymphoid cells may include 1,000 lymphoid cells. For example, the plurality of lymphoid cells may include 1,000 to 2,000, 2,000 to 5,000, 5,000 to 10,000, or more than 10,000 lymphoid cells.

The data from the image may include at least 100,000 pixels, including, for example, 100,000 to 250,000, 250,000 to 500,000, 500,00 to 1 million, 1 to 2 million, 2 to 5 million, 5 to 10 million, or more than 10 million pixels. The pixel may be two-dimensional. In some embodiments, the pixel may be three-dimensional (i.e., a voxel). The data may include values for color intensities of each pixel. The color intensities may be for each color channel of a plurality of color channels. For example, the plurality of color channels may be RGB or another color triplet. In some embodiments, the data may include values for grayscale intensity. The data of each pixel may include coordinates of the pixel relative to the image. In some embodiments, the data of each pixel may include coordinates relative to a location within a slide or within a tissue.

In some embodiments, process 700 may include obtaining the biological sample from the subject. The biological sample may be obtained as a biopsy. The biopsy may be a needle core biopsy or an excisional biopsy. The biological sample may be from a lymph node. In these and other embodiments, process 700 may include capturing the image of the plurality of lymphoid cells in the biological sample by performing microscopy on the biological sample. Microscopy may be by digital microscopy, e.g., Aperio AT2 brightfield scanner.

The data may be from a region of interest of an image. The image may be a whole slide image. The image may include the plurality of lymphoid cells and an additional plurality of lymphoid cells. A region including the plurality of lymphoid cells and not including the additional plurality of lymphoid cells may be selected as described herein. The region may be selected to include the predominant shape or size of cell in the image. The region may be selected for an area that appears to have lymphoma or leukemia, using a pathologist's judgment. The image may be divided into a number of regions (e.g., a grid), and an image quality score may be evaluated for each region. The image quality score may be determined using the color intensity, sharpness, contrast, and/or other image characteristics of the region. Additionally, the image quality score may also include a determination of the relevance of the particular tissue location to the classification of leukemia or lymphoma levels. The region of interest may be selected from regions having a minimum image quality score. In some embodiments, the selection of the region of interest may be by a hemapathologist. In other embodiments, the selection of the region of interest may be by a computer. The region may have a minimum width and height of 500, 600, 700, 800, 900, 1,000, 1,500, or 2,000 pixels. The selection of the region may be performed prior to blocks 720-750.

In some embodiments, the data may be normalized. For example, the data may be normalized based on a maximum intensity value. The data may include an intensity value for each color channel. Each color channel may be normalized based on one or more maximum intensity values. In some embodiments, pixels with outlier intensity values may be replaced with the maximum intensity value.

At block 720, first pixels corresponding to the plurality of lymphoid cells may be identified in the data. The identification of the plurality of lymphoid cells may include analyzing color intensity of pixels in the data. Pixels with a color intensity matching or within a certain range of the expected color intensity of a stained cell may be identified as corresponding to a lymphoid cell. Continuous areas of pixels with the requisite color intensity may be grouped together as a single cell.

At block 730, each lymphoid cell of the plurality of lymphoid cells may be segmented into second pixels corresponding to a first portion with a nucleus and third pixels corresponding to a second portion without a nucleus. The second pixels and third pixels are subsets of the first pixels. The segmentation may use the first pixels corresponding to the plurality of lymphoid cells. The second portion may be the cytoplasm portion of the cell. The segmentation may involve determining that intensities the third pixels are closer to a reference intensity for stained nuclei than a reference intensity for cytoplasm of the cell. In some embodiments, the segmentation may involve analyzing contrast between neighboring pixels, where areas of high contrast indicate a boundary between a nucleus and the cytoplasm. Segmentation may be by a machine learning model, including a convolutional neural network. Segmentation may be performed using the Hover-Net model.

At block 740, the plurality of lymphoid cells may be filtered, using the second pixels and the third pixels, to remove lymphoid cells having overlapping first portions to produce fourth pixels corresponding to a filtered plurality of lymphoid cells. The fourth pixels are subsets of the first pixels. In some embodiments, filtering the plurality of lymphoid cells may be optional and the plurality of lymphoid cells may be used rather than the filtered plurality of lymphoid cells. Filtering the plurality of lymphoid cells may include determining a ratio of the first portion with the nucleus and the second portion without the nucleus. Determining the ratio may involve the number of third pixels and the number of fourth pixels. In some embodiments, the ratio determination may be for each lymphoid cell of the plurality of lymphoid cells. As an example, the ratio may be the number of third pixels in the cell divided by the number of fourth pixels in the cell. In other embodiments, the process may include determining which cells have a nuclear portion having a concave section in the boundary. The ratio may be calculated for only cells having the concave section in the boundary. The ratio may be a solidity calculation as described herein. The ratio may be compared to a threshold value. The lymphoid cell may be determined to be in the filtered plurality of lymphoid cells when the ratio exceeds the threshold value.

In some embodiments, filtering may include determining the size of the nuclei. The filtered cells may include only cells with nuclei in a certain size range. For example, the nuclei may be represented by pixels corresponding to sizes from 8 μm² to 108 μm², 5 μm² to 10 μm², 10 μm² to 50 μm², 50 μm² to 80 μm², 80 μm² to 100 μm², or 100 μm² to 110 μm².

At block 750, a value for nuclear size, a value for nuclear intensity, and a value for cellular density may be measured using the fourth pixels corresponding to the filtered plurality of lymphoid cells. In some embodiments, blocks 720, 730, and 740 may be optional. The values may be measured from data of an image captured of the plurality of lymphoid cells. Pixels corresponding to nuclei of the plurality of lymphoid cells may be identified. These pixels may then be used for measurements.

In some embodiments, measuring the value for nuclear size may include measuring a cellular value for the nuclear size for each lymphoid cell of the filtered plurality of lymphoid cells. The cellular value may be compared to a cutoff value. The cutoff value may be 24 μm², from 20 to 25 μm², from 25 to 30 μm², or from 30 to 40 μm². The lymphoid cell may be determined whether to be in a size classification using the comparison. The size classification may be large cells (e.g., cells larger than the cutoff value). An amount of lymphoid cells in the size classification may be calculated. The amount of lymphoid cells in the size classification may be a ratio determined using a number of lymphoid cells in the size classification and a number of lymphoid cells not in the size classification. For example, the ratio may be a ratio of the number of cells with nuclei larger than the cutoff value to the number of cells with nuclei smaller than or equal to the cutoff value. In some embodiments, the ratio may be the number of cells with nuclei larger than the cutoff value to all cells.

The cutoff value may be determined using an objective function. The objective function may be determined using amounts of lymphoid cell types in a training data set. The objective function may be as described herein. For example, a known type (e.g., CLL, aCLL, and RT) of cell may have its respective ratio calculated based on different cutoff values. The objective function may be based on a separation value in the ratios between any two known types of the different types. For example, the ratio for CLL, aCLL, and RT may be represented as R_{CLL}, R_{acLL}, and R_{RT}, respectively. The objective function may be based on the differences between the ratios. For example, the objective function may be (R_{RT}−R_{acLL})*(R_{acLL}−R_{CLL})*(R_{RT}−R_{CLL}). In other embodiments, the objective function may be based on ratios between the ratios. For example, the objective function may be (R_{RT}/R_{acLL})*(R_{acLL}/R_{CLL})*(R_{RT}/R_{CLL}). The cutoff value may be a value that maximizes the objective function or results in an objective function above a minimum reference value. The minimum reference value may be based on objective functions determined using cells in reference data sets.

In some embodiments, measuring the value for nuclear intensity may include measuring subvalues for a plurality of color channels. For example, a first subvalue may be measured for a red channel, a second subvalue may be measured for a green channel, and a third subvalue may be measured for a blue channel. Each subvalue may be a statistical value of all values measured for the filtered plurality of lymphoid cells. For example, the first subvalue for the red channel may be a mean, median, mode, or percentile of intensity values. A statistical value of the subvalues for the plurality of color channels may be determined. The statistical value may be a mean or median of the subvalues for the plurality of color channels. In other embodiments, the value for nuclear intensity may include measuring intensity based on grayscale (either white or black represent maximum intensity).

Cellular density may be measured by determining the number of cells per area (e.g., per μm², per image, or per region of interest). In some embodiments, cellular density may be measured by determining the number of pixels corresponding to the nuclei of the filtered plurality of lymphoid cells per area.

In some embodiments, a value for distances between cells may be measured. Measuring the value for distances between cells may include measuring the distances between each cell and the nearest neighbor of each cell. The nearest neighbor may be determined to be the neighbor cell with the shortest distance from the cell. In some aspects, measuring the value for distances between cells may include measuring a value for distances between nuclei. For example, the distance may be between each nuclei and the nearest nuclei. In other aspects, measuring the value for distances between the nuclei may include determining a centroid for each nucleus of each lymphoid cell of the filtered plurality of lymphoid cells. Measuring the value for distances between cells may include measuring a value for distances between centroids (e.g., each centroid and its nearest centroid). The value for distances between cells may be a statistical value (e.g., mean, median, mode, percentile) of distances measured between cells.

At block 760, a classification of leukemia or lymphoma in the subject may be determined using the measured values. In some embodiments, the classification of leukemia or lymphoma may use no values measured from the filtered plurality of lymphoid cells other than the value for nuclear size, the value for nuclear intensity, the value for cellular density, and the value for distances between cells. In some embodiments, the classification may be determined without cell morphologic or cytologic traits other than nuclear size and nuclear intensity. In some embodiments, the classification may be determined without spatial pattern or architectural parameters other than cellular density and cellular distance. Measurements that may be excluded from determining classification may include variance/skewness/kurtosis of the cell size and intensity; average/variance/skewness/kurtosis of the cell length/width ratio; and average/variance/skewness/kurtosis of the cell circularity.

In some embodiments, determining the classification of leukemia or lymphoma comprises determining a level of chronic lymphocytic leukemia (CLL), accelerated CLL (aCLL), or Richter transformation (RT). Determining the classification may include determining a probability that each classification of CLL, aCLL, or RT exists. The classification may be determined based on the classification with the highest probability. The classification may require the probability to be above a reference probability (e.g., 50%, 60%, 70%, 80%, 90%, 95%, or 99%). The level may be the probability that CLL, aCLL, or RT exists, or the level may be whether CLL, aCLL, or RT exists. The classification may be a level of non-Hodgkin's lymphoma (CLL, aCLL, and RT fall under non-Hodgkin's lymphoma). In some embodiments, the classification may be determining a level of a normal, healthy condition (i.e., without leukemia or lymphoma). In some embodiments, the classification is of Hodgkin's lymphoma (e.g., presence or different severity or levels of Hodgkin's lymphoma).

Determining the classification may be through using a machine learning model. The plurality of lymphoid cells may be a first plurality of lymphoid cells. The value for nuclear size may be a first value for nuclear size. The value for nuclear intensity may be a first value for nuclear intensity. The value for cellular density may be a first value for cellular density. Determining the classification may use a machine learning model trained by receiving a plurality of training images. Each training image may include a second plurality of lymphoid cells. Each training image of the plurality of training images may be labeled with a known classification of leukemia or lymphoma. The known classification may be CLL, aCLL, or RT. In some embodiments, the known classification may include a normal, healthy condition. For each training image of the plurality of training images, a second value for nuclear size, a second value for nuclear intensity, and a second value for cellular density may be measured. In some embodiments, a second value for distances between cells may be measured. The machine learning model may be trained by optimizing parameters of the machine learning model based on outputs of the machine learning model matching or not matching the known classification when the second values are input into the machine learning model. An output of the machine learning model may specify a classification of leukemia or lymphoma for the training image. In some embodiments, process 700 includes training the machine learning model using the plurality of training images.

The machine learning model may include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm The machine learning model may also include other machine learning models, including convolutional neural networks (CNN), linear regression, logistic regression, deep recurrent neural network (e.g., long short-term memory, LSTM), Bayes classifier, hidden Markov model (HMM), linear discriminant analysis (LDA), k-means clustering, density-based spatial clustering of applications with noise (DBSCAN), random forest algorithm, and support vector machine (SVM).

The classification of leukemia or lymphoma may be that the subject has leukemia or lymphoma. For example, the classification may be that any of CLL, aCLL, or RT exists, and leukemia or lymphoma may be determined to exist. In some embodiments, the classification of a disease may be determined in combination with considering clinical data of the subject, including symptoms of lymphoma.

In some embodiments, process 700 may include comparing the classification with a previous classification of leukemia or lymphoma in the subject. The previous classification may occur at a time at least 1 day, 1 week, 1 month, or 1 year before the current classification. In some embodiments, the previous classification may be before a treatment, a change in treatment, or surgery, and the current classification may be after the treatment, a change in treatment, or surgery. The process may include determining a progression of the leukemia or lymphoma in the subject based on the comparing. For example, the previous classification may be a less severe classification, where the classification is more severe from normal to CLL to aCLL to RT. A more severe classification compared to the previous classification may result in a more aggressive treatment plan, which may include a higher dose or frequency of treatment. A less severe classification compared to the previous classification may result in a less aggressive treatment plan, which may include a lower dose or frequency of treatment.

The method may further include treating the subject. Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

Process 700 may include additional implementations, such as any single implementation or any combination of implementations described and/or in connection with one or more other processes described elsewhere herein.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

### B. Proliferation Center Mapping

Chronic lymphocytic leukemia/small lymphocytic lymphoma (CLL) is characterized morphologically by numerous small lymphocytes and pale nodules composed of prolymphocytes and paraimmunoblasts known as proliferation centers (PCs). Distinguishing PCs in CLL from aCLL or RT can be diagnostically challenging, particularly in small needle-biopsy specimens. Available guidelines pertaining to distinguishing of CLL from its progressive forms are limited, subject to the morphologist's experience and are often not completely helpful in the assessment of scant biopsy specimens. To objectively assess the extent of PCs in aCLL and RT, and enhance diagnostic accuracy, we sought to design an artificial intelligence (AI)-based tool to identify and delineate PCs based on feature analysis of the combined individual cell size and intensity, designated here as the heat value. Using the mean heat value from the generated heat value image of all cases, we were able to reliably separate CLL, aCLL, and RT with robust diagnostic predictive values.

Lymph nodes involved by chronic lymphocytic leukemia/small lymphocytic lymphoma (CLL/SLL) are characterized by replacement of nodal architecture by a dominant infiltrate of small lymphocytes interspersed by areas termed proliferation centers (PC). The PC areas are composed of prolymphocytes and paraimmunoblasts that have increased mitotic activity. While most CLL patients have an indolent clinical course, a subset can develop more aggressive disease, either “accelerated phase of CLL/SLL” (aCLL) or Richter transformation (RT). Histologically, lymph nodes with aCLL have an increased number and size of proliferation centers, which become confluent (by definition, broader than a 20× field). These changes also entail an increased Ki67 proliferation index (>40% in PCs) and mitotic figures (>2.4 mitoses/PC). A subset of CLL patients, with or without detectable aCLL, develop RT. RT is characterized by confluent growth of large cells, occasionally interspersed by cells indicative of remnant CLL. Patients who develop RT have a poor prognosis, with a median overall survival of <12 months despite intensive chemoimmunotherapy.

Although data are scant regarding the clinical outcomes of patients with accelerated CLL/SLL, these data suggest that patients with aCLL have poorer outcomes than patients with CLL/SLL. Nevertheless, the current World Health Organization (WHO) classification of hematolymphoid neoplasms does not provide morphologic guidelines to assess CLL cases with clinical suspicion of disease acceleration. In addition, literature pertaining to this topic is very limited, and identifying features of disease acceleration based on available limited guidelines (PCs broader than a 20× field, Ki67 proliferation index>40% in PCs and mitotic figures>2.4 mitoses/PC) are subjective and depend on hematopathologist's experience, especially in scant tissue biopsy samples.

The application of computer-aided diagnostic algorithms based on clinically interpretable models may provide a much-needed assistance in this well-defined clinical scenario. In this study, we sought to design an AI-based tool that can provide an objective assessment to understanding low-power magnification architectural changes, and for enhancing the delineation of PCs in CLL, and in its accelerated and transformed phases. To this aim, we have performed a combined “cell-size and intensity analysis” that we termed “heat value”. Using the mean heat value from the generated heat value image of all cases, we were able to reliably separate the three phases in question with robust diagnostic predictive values.

### 1. Material and Methods

### a) Data Collection

The study was approved by The University of Texas MD Anderson Cancer Center (UTMDACC) Institutional Review Board. We retrospectively searched patients with hematolymphoid diseases clinically evaluated at UTMDACC between Feb. 1, 2009, and Jul. 31, 2021. We randomly selected 10 CLL, 12 aCLL, and 8 RT digitized hematoxylin and eosin stained slides of excisional biopsy specimens to study the mapping of proliferation centers. All selected slides came from different patients, and in total we manually annotated 25, 28, and 21 regions of interest (ROI) encompassing small round PCs and confluent/expanded PCs from CLL, aCLL, and RT, respectively. To ensure the heatmap generated from mapping of proliferation centers had sufficient information, both the length and width of the annotated ROI were set at larger than 2,000 pixels. Stain normalization was performed on all ROI prior to further processing.

### b) Cell Segmentation and Refinement

This study aimed to objectively automate mapping of PCs, which is visually delineated by pathologists in clinical practice during evaluation of glass slides. Our proposed mapping model is based on visual properties of individual cells (size and intensity), thus cell segmentation is a prerequisite. We employed Hover-Net for the cell segmentation task, given that Hover-Net is a state-of-the-art cell segmentation algorithm pre-trained on the MoNuSeg dataset, thus avoiding time-consuming cell annotation and model tuning procedures.

Nevertheless, after Hover-Net segmentation, inevitably we encountered overlapping cells that were inaccurately segmented. To address this issue, we deployed the solidity feature, defined as the ratio of segmented cell contour area to its convex hull area, to filter out overlapping cells. Segmented cells with a solidity value smaller than 0.84 were removed from further analysis. In addition, we set the minimum and maximum pixel number of the cell to be 32 and 432, which corresponds to 8 μm² and 108 μm², respectively. We also discarded cells with a pixel number outside the set range.

### c) Automated Mapping of Proliferation Centers

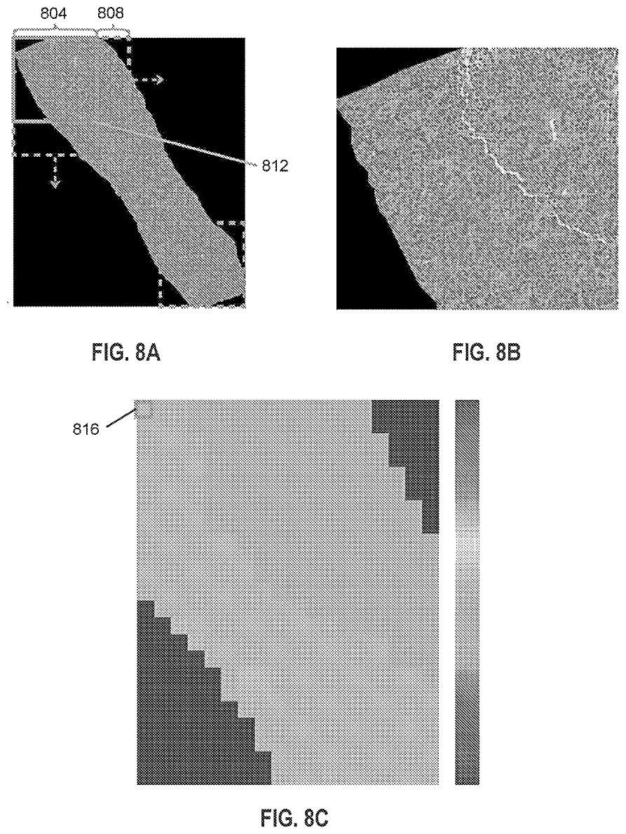

FIG. 8A shows splitting each ROI into multiple tiles. The ROI may be manually annotated, followed by nuclear segmentation and filtering, as described herein. The tiles are determined by the tile length 804 and stride 808. These two parameters are set to be the same on both horizontal and vertical directions and across all ROI. In our experiments, we set the tile length and stride as 1,000 and 100 pixels, respectively. As the value of tile length is larger than the stride, some neighboring tiles overlapped, thus creating a sufficient number of tiles to map PCs. Tile 812 is an example of a tile with tile length 804. FIG. 8B shows a magnified image of tile 812 with tile length 804. The white line within the image is an artifact of processing.

We then conducted feature analysis of the combined size/intensity properties of nuclei inside each tile, to generate and recreate a novel representation of PCs. As nuclear size varied from 8 to 108 square micrometers, and nuclear mean intensity varied from 0 to 255, we normalized the values of nuclear size and mean intensity to 0.0 and 1.0, by subtracting the minimum value and dividing it by the value range length, and represented as S(nucleusᵢ) and Iₘₑₐₙ(nuceuslᵢ), respectively. We then estimated the heat value of each tile by integrating nuclear size and mean intensity using the following formula:

$$\frac{1}{{N}_{nucleus}} {\sum }_{i = 1}^{{N}_{nucleus}} \sqrt{S ( {nucleus}_{i} ) * {I}_{mean} ( {nucleus}_{i} )} .$$

With the proposed heat value estimation formula, we calculated the heat value for each tile in each ROI. We then generated a heat value image for each ROI to map its PCs. FIG. 8C shows the heat value image. Area 815 reflects the heat value for tile 812 from FIG. 8A and FIG. 8B. Dark blue indicates a heat value of 0. Dark red indicates a heat value of 1. In FIG. 8, the more yellow the color, the higher the heat value. A higher heat value corresponds to increased nuclear size and mean intensity. Each tile is represented as a pixel even though the tiles overlap. We repeated this process for ROI of all CLL, aCLL, and RT cases.

Based on our proposed heat value formula, the heat values in the heat value image were less than 0.5 in analyzed ROIs. Instead of directly converting the heat value image to the heatmap, we first accentuated heat values by multiplying by a factor of 2.0. Then we performed heatmap conversion based on the scaled heat value image for better visualization. We applied Otsu's method to identify the optimal threshold for each heat value image and regarded the segmented foreground as PC areas.

For an objective quantification of the heat value image, we went a step further and generated a heat value histogram for each heat value image. Based on the obtained histograms, we employed the F-score (a measure of a test's accuracy using the following formula: F-score=2.0×(precision×recall)/(precision+recall)) to identify two heat value thresholds and achieve an optimal separation performance among the three entities. The two-sided test was used to quantify the difference between diseases.

### 2. Generating Heat Values and Heatmaps

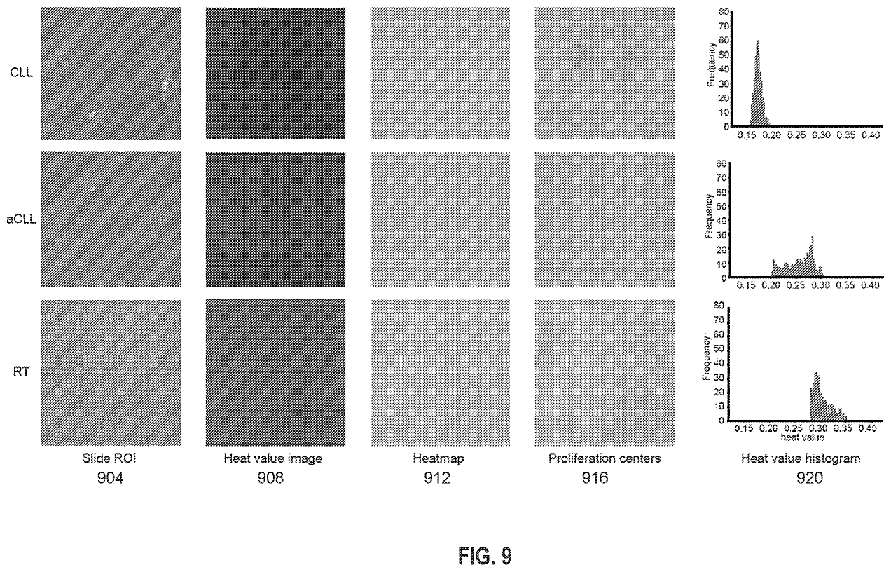

FIG. 9 shows different images of the region of interest and histograms of the heat values for the ROIs of CLL, aCLL, and RT disease phases. Column 904 shows raw slide images of the ROI. Column 908 shows a grayscale image reflecting the heat values of the tiles in the ROI. Column 912 shows a heatmap after the heat values in the images of column 908 were accentuated to sharpen color separation.

Column 916 shows the heatmap after identifying proliferation centers (PCs). Areas with high heat values (in the yellow spectrum) correspond to tiles harboring cells with increased nuclear size and mean intensity (PCs in CLL cases and expanded/confluent PCs in aCLL and RT cases). In contrast, areas with low heat values (in the blue spectrum) correspond to tiles with decreased nuclear size and mean intensity, representing small neoplastic lymphocytes surrounding PCs. This recreation of PCs based on objective measures of cellular attributes (size and intensity) provides on its own a visual aid to assess the extent of large cells depicted in yellow in relation to small neoplastic lymphocytes in blue, in the three disease phases: Yellow foci confined in small PC, and occupying a subset of the ROI, with predominantly blue areas composed of small-size neoplastic lymphocytes with decreased intensity in CLL; Expanding yellow foci creating confluent PCs, with decreasing background blue areas in aCLL; Predominantly yellow ROI with sheets of large cells, resulting from fusing of PCs, and virtually absent blue areas in RT.

Column 920 shows histograms of the heat values from the heat value images (column 908). Based on the histograms, we isolated two heat value thresholds based on the F-score to achieve separation among the three disease phases: 0.228, below which the case was most likely to be CLL (the top histogram in column 920 represents a CLL ROI with heat values ranging between 0.16 and 0.19, and peaking at 0.18 approximately); and 0.288, above which the case was most likely to be RT (the bottom histogram in column 920 represents an RT ROI with heat values ranging between 0.20 and 0.30, and peaking at 0.27 approximately); Cases with heat values ranging between 0.228 and 0.288 were most likely aCLL (the middle histogram in column 920 represents an aCLL ROI with heat values ranging between 0.28 and 0.35, and peaking at 0.29 approximately).

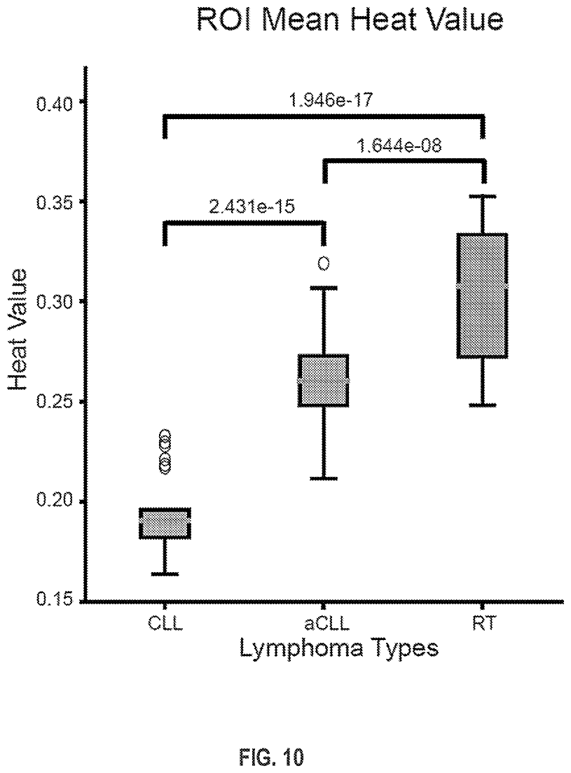

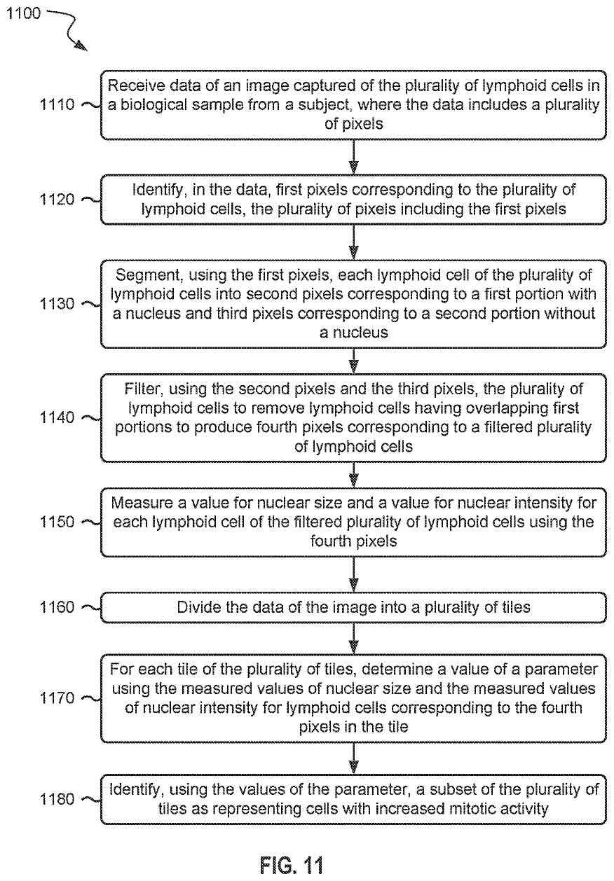

FIG. 10 shows a boxplot of the mean heat value frequencies of all ROI from the three phases. The x-axis shows the lymphoma types. The y-axis shows the heat value. There was a significant difference in the ranges of mean heat value frequencies for CLL, aCLL, and RT, which were 0.168 to 0.233, 0.212 to 0.307, and 0.261 to 0.353, respectively. The p value between CLL and aCLL was 2.431e−15. The p value between aCLL and RT was 1.644e−06. The p value between CLL and RT was 1.946e−17. The accuracy and area under the curve diagnostic predictive values using data from nuclear size alone were 0.658 (+/−0.115) and 0.771 (+/−0.096), respectively; and using mean nuclear intensity alone, 0.679 (+/−0.094) and 0.841 (+/−0.052), respectively; with a noticeable increase using heat value frequencies (integrating the nuclear size and mean nuclear intensity) reaching 0.813 (+/−0.0630) and 0.885 (+/−0.109), respectively.

### 3. Accuracy in Distinguishing Disease Phases

Clinically, accelerated phase disease behaves similarly to typical CLL in terms of B-symptoms, disease bulk, functional status and clinical stage, these patients often have higher serum LDH levels and are ZAP70 positive. Some data suggest that the prognosis of aCLL patients is poorer than that of CLL patients. In contrast, patients with unequivocal disease transformation, or RT, are notoriously more symptomatic, have lower performance status, higher serum LDH levels, and higher uptake on PET-CT scan. Although in some practices, aCLL cases are still treated like CLL (combination therapy of ibrutinib and venetoclax), switching to a more intensive treatment regimen for patients with aCLL in some settings results in a better clinical response, especially in CLL patients who become refractory to treatment (such CLL patients may harbor undiagnosed aCLL). Thus, distinguishing classic CLL from its accelerated phase morphologically is important, with aCLL possibly indicating disease progression and the need to upgrade treatment.

Hematopathologists rely on low magnification microscopic examination to characterize the shape of PCs in patients with history of CLL. Small round and distinct PCs are indicative of an underlying CLL, whereas confluent PCs occupying larger areas are more indicative of underlying disease acceleration (aCLL). Lastly, expanded sheets of large cells, beyond a recognizable PC morphology, is diagnostic of RT. Analysis of PC expansion/formation of sheets of large cells is conducted based on the assessment of H&E glass slide at low magnification, coupled with Ki67 stain that may highlight the extent of large cell (˜ mitotically active cell) expansion. Expanded proliferation centers in aCLL may be considered to be broader than a 20× field. However, in our experience, this assessment is morphologist-dependent and varies greatly depending on the exposure of hematopatologists to these particular cases.

As discussed herein, we developed an artificial intelligence-based “disease diagnosis model” in which we isolated “cellular morphologic features” that we implemented as biomarkers to enhance diagnostic accuracy in determining CLL, aCLL, and RT. In this section, we sought to design an “architecture-based” tool to enhance the delineation of PCs, by implementing a novel technique that integrates nuclear size and intensity. By applying this tool, large nuclei with high intensity, and small nuclei with low intensity occupy the yellow and blue spectra, respectively (e.g., columns 912 and 916 in FIG. 9). Using this method, we were able to enhance the visualization of large nuclear overall architectural distribution and extent in studied ROI: confined yellow foci in PCs in CLL, confluent yellow foci representing fused PCs in aCLL, or yellow sheets replacing the vast majority of the ROI in RT (column 916 in FIG. 9). As we were able to reproduce these results across ROI from the three disease phases, this heat value method may aid in the visual assessment and/or diagnosis of challenging cases with features of disease acceleration/transformation, especially in limited core-needle biopsy specimens.

In addition to visually mapping the extent of large nuclei, we plotted the heat values of all tiles to their frequencies per ROI (column 920 in FIG. 9). By repeating this process in all ROIs, we isolated two heat value coefficients: 0.228, below which a case is most likely to be classified as CLL; and 0.288, above which a case is most likely to be classified as RT. Cases lying in between these two coefficients are most likely to be aCLL. We also noticed that heat value frequencies in CLL ROI had a single Gaussian-like prominent peak, as illustrated in the example we provide in the top histogram in column 920 in FIG. 9. Heat value frequencies that demonstrated a smearing pattern, with no definitive dominating peak in aCLL cases, are illustrated in the example we provide in the middle histogram in column 920 of FIG. 9. Finally, RT cases demonstrated a right-shifted distribution of heat value distribution frequency, with some tiles occupying increasing heat values, as illustrated in the example we provide in the bottom histogram in column 920 of FIG. 9.

To test the generalizability of the findings described above, we plotted the mean heat value in all ROIs across the three disease phases in FIG. 10. The range of mean heat value frequencies demonstrated a statistically significant separation among the three phases in most cases, with one aCLL case overlapping with RT range, and a few CLL cases overlapping with aCLL range. Overlapping ranges among entities on rare occasions could be attributed to ROI sampling of active PCs belonging to the far end side of disease spectrum, as PCs in CLL and aCLL are dynamic environments and could be captured at any point during their growth, including phases in which a minor percentage of them have crossed over to a more progressive state (CLL into aCLL or aCLL into RT).

Our data suggest that this model, based on objective architectural analysis of PCs, is able to achieve a robust diagnostic accuracy. Although our design was performed on excisional biopsy specimens, which are inherently more informative morphologically, the end goal of this model is to deploy it in limited biopsy specimens. In fact, core-needle biopsy is nowadays a more common method of tissue sampling in the setting of clinical suspicion of underlying disease progression/transformation, as these specimens can be obtained more rapidly and are minimally invasive in comparison to excisional biopsies. However, core-needle biopsy specimens provide an incomplete picture of the underlying nodal architecture, a keystone in the assessment of accelerated disease, and delivering an accurate and confident diagnosis in this challenging scenario may be achieved by the assistance of objective tools. We suggest that our model, with further refinement and sophistication, can be ultimately deployed to this aim.

Embodiments provide an architecture-based tool to objectively assess the extent of PCs in CLL cases with clinical suspicion of disease progression, based on the integrative analysis of cell nuclear size and mean nuclear intensity and automation of PC mapping. Using the mean heat value of all cases, we were able to reliably separate the three disease phases in question with robust diagnostic predictive values. In these examples, an ROI mean heat value less than 0.228 is predictive of CLL, and a value more than 0.288 is predictive of RT. aCLL cases demonstrate a mean heat value ranging from 0.228 to 0.288. This work highlights the value of using artificial intelligence-based tools in identifying clinically meaningful cellular and architectural features, to enhance disease diagnosis in challenging clinical scenarios. Our model, although trained and tested on excisional biopsy specimens, could be potentially very useful in the assessment of limited core-needle biopsy specimens, where typically only a small percentage of PCs is available for morphologic evaluation of architecture and extent of growth.

### 4. Example Methods

### a) Tissue Visualization Using Heat Values

FIG. 11 is a flowchart of an example process 1100 associated with methods and systems for determining leukemia or lymphoma levels using lymphoid images. In some implementations, one or more process blocks of FIG. 11 may be performed by a system (e.g., system 2500). In some implementations, one or more process blocks of FIG. 11 may be performed by another device or a group of devices separate from or including the system. Additionally, or alternatively, one or more process blocks of FIG. 11 may be performed by one or more components of system 2500, such as imaging device 2510, detector 2520, logic system 2530, processor 2550, memory 2535, external memory 2540, storage device 2545, and/or treatment device 2560.

At block 1110, data of an image captured of the plurality of lymphoid cells in a biological sample from a subject is received. The data includes pixels. Block 1110 may be performed in the same or similar manner as described with block 710.

At block 1120, first pixels in the data corresponding to the plurality of lymphoid cells are identified. Block 1120 may be performed in the same or similar manner as described with block 720.

At block 1130, each lymphoid cell of the plurality of lymphoid cells is segmented, using the first pixels, into second pixels corresponding to a first portion with a nucleus and third pixels corresponding to a second portion without a nucleus. Block 1130 may be performed in the same or similar manner as described with block 730.

At block 1140, the plurality of lymphoid cells is filtered, using the second pixels and the third pixels, to remove lymphoid cells having overlapping first portions to produce fourth pixels corresponding to a filtered plurality of lymphoid cells. Block 1140 may be performed in the same or similar manner as described with block 740. In some embodiments, filtering the plurality of lymphoid cells may be optional and the plurality of lymphoid cells may be used rather than the filtered plurality of lymphoid cells.

At block 1150, a value for nuclear size and a value for nuclear intensity is measured for each lymphoid cell of the filtered plurality of lymphoid cells using the fourth pixels. In some embodiments, blocks 1120, 1130, and 1140 may be optional. Measuring the value for nuclear size and the value for nuclear intensity may be performed as described with block 750.

At block 1160, the data of the image may be divided into a plurality of tiles. The plurality of tiles may be the same size or different sizes. Each tile may have the same dimensions (e.g., length and width). Each tile of the plurality of tiles may overlap with at least one other tile of the plurality of tiles. In some embodiments, the plurality of tiles is non-overlapping. The plurality of tiles may be determined by one or more dimensions of the tiles and a stride. The stride determines the offset of one tile from the other tile. The length and/or width of a tile may be from 100 to 500, 500 to 600, 600 to 700, 700 to 800, 800 to 900, 900 to 1,000, 1,000 to 1,500, 1,500 to 2,000, 2,000 to 5,000, or over 5,000 pixels. The stride may be from 10 to 50, 50 to 100, 100 to 200, 200 to 500, 500 to 1,000, or over 1,000 pixels.

At block 1170, for each tile of the plurality of tiles, a value of a parameter is determined using the measured values of nuclear size and the measured values of nuclear intensity for lymphoid cells corresponding to the fourth pixels in the tile. The value of the parameter may be a heat value described herein. The number of lymphoid cells in each tile of the plurality of tiles may be determined. The value of the parameter may be determined using the formula:

$$\frac{1}{{N}_{nucleus}} {\sum }_{i = 1}^{{N}_{nucleus}} \sqrt{S ( {nucleus}_{i} ) * {I}_{mean} ( {nucleus}_{i} )}$$

where N_{nucleus} is the number of nuclei, i is an integer, S(nucleusᵢ) is the size of nucleusᵢ, and Iₘₑₐₙ(nucleusᵢ) is the mean nuclear intensity of nucleusᵢ. In some embodiments, a statistical value (e.g., mean, median, mode, percentile) of the nuclear intensity of nucleusᵢ may be used in determining the parameter. In some embodiments, the parameter may include cellular density and/or cell-to-nearest neighbor distance. In some embodiments, the parameter may include or exclude the same measurements from the filtered plurality of lymphoid cells that may be excluded in block 760.

In embodiments, these and other parameters are possible. The parameter may use the nuclear size and the nuclear intensity. The parameter may include a product or sum of the measurements (e.g., nuclear size, the nuclear intensity). The parameter may include a geometric and/or arithmetic mean of the measurements. The parameter may represent an averaged value for a nucleus.

At block 1180, a subset of the plurality of tiles is identified as representing cells with increased mitotic activity using the values of the parameter. The subset of the plurality of tiles may represent one or more proliferation centers (PCs).

Process 1100 may include additional implementations, such as any single implementation or any combination of implementations described herein and/or in connection with one or more other processes described herein.

In a first implementation, identifying the subset of the plurality of tiles comprises for each tile of the plurality of tiles comparing the value of the parameter with a threshold value, and determining the tile to be in the subset when the value exceeds the threshold value. Identifying the subset of the plurality of tiles may include identifying the foreground of an image. In some embodiments, the threshold value is determined by Otsu's method.

In some embodiments, process 1100 includes determining a classification of leukemia or lymphoma in the subject using the subset of the plurality of tiles. Determining the classification of leukemia or lymphoma may include determining a level of chronic lymphocytic leukemia (CLL), accelerated CLL, or Richter transformation (RT). In embodiments, determining the classification of leukemia or lymphoma in the subject may include determining a statistical value of the values of the parameter for the subset of the plurality of tiles, and comparing the statistical value to a cutoff value. In some embodiments, the statistical value may be a mean, median, mode, or percentile of the values. In some embodiments, the statistical value may characterize the type of distribution. The statistical value may indicate how Gaussian, uniform, or shifted the distribution is. A more Gaussian distribution may indicate CLL. A more uniform or smeared distribution may indicate aCLL. A right-shifted distribution may indicate RT. Examples of distributions are shown in column 920 of FIG. 9.

In some embodiments, determining the classification of leukemia or lymphoma in the subject may include determining an amount of tiles in the subset of the plurality of tiles relative to the plurality of tiles, and comparing the amount to a cutoff amount. For example, a proportion of tiles that are in the subset of the plurality of tiles may be determined. The amount may indicate how large the proliferation centers are (see column 916 in FIG. 9). A lower amount or proportion may indicate CLL. A moderate or higher amount may indicate aCLL or RT.

The cutoff value or cutoff amount may divide one classification from another classification. The cutoff value or cutoff amount may be determined from one or more reference samples with a known classification. For example, most or all reference samples with RT may have a statistical value for the respective subset of tiles above (or below) a certain threshold or in a certain range. Similarly, reference samples with CLL may have a statistical value for the respective subset of tiles below (or above) a certain threshold or in a certain range. Additionally, reference samples with aCLL may have a statistical value for the respective subset of tiles below or above a certain threshold or in a certain range. The threshold or an end of the range may be used as a cutoff value. Cutoff values may be determined as described with column 920 of FIG. 9 and FIG. 10.

In some embodiments, an image including fifth pixels may be generated. The intensity of each fifth pixel of the fifth pixels may be determined using the value of the parameter for a tile of the subset of the plurality of tiles. The image with fifth pixels may be a heat value image (column 908), a heatmap (column 912), or a proliferation center image (column 916). The intensity of the fifth pixels may be the value of the parameter (e.g., the heat value) or a scaled value of the parameter (e.g., multiplied by a factor). The image of fifth pixels may be displayed.

In embodiments, process 1100 may include determining a shape classification of the subset of the plurality of tiles. The classification of leukemia or lymphoma in the subject may be determined using the shape classification. A shape classification being small, round, and/or distinct may indicate CLL. A shape classification of confluent may indicate aCLL. A shape classification of large and expanded sheets may indicate RT. The shape classification may be determined by a medical practitioner (e.g., a hemapathologist) or a computer system. The shape classification may be determined using measurements, such as size, aspect ratio, area-to-perimeter ratio. A certain shape classification can be determined when one or more of the measurements is greater than or less than a cutoff value or within a certain range. The cutoff value or range may be determined from reference samples with known shape classifications or known levels of leukemia or lymphoma.

The classification of leukemia or lymphoma in the subject may be used as described with process 700. The classification may be compared to classifications at other times. A treatment may be determined and administered based on the classification or the change in classification.

Although FIG. 11 shows example blocks of process 1100, in some implementations, process 1100 may include additional blocks, fewer blocks, different blocks, or differently arranged blocks than those depicted in FIG. 11. Additionally, or alternatively, two or more of the blocks of process 1100 may be performed in parallel.

### b) Classification Using Heat Values of Tiles

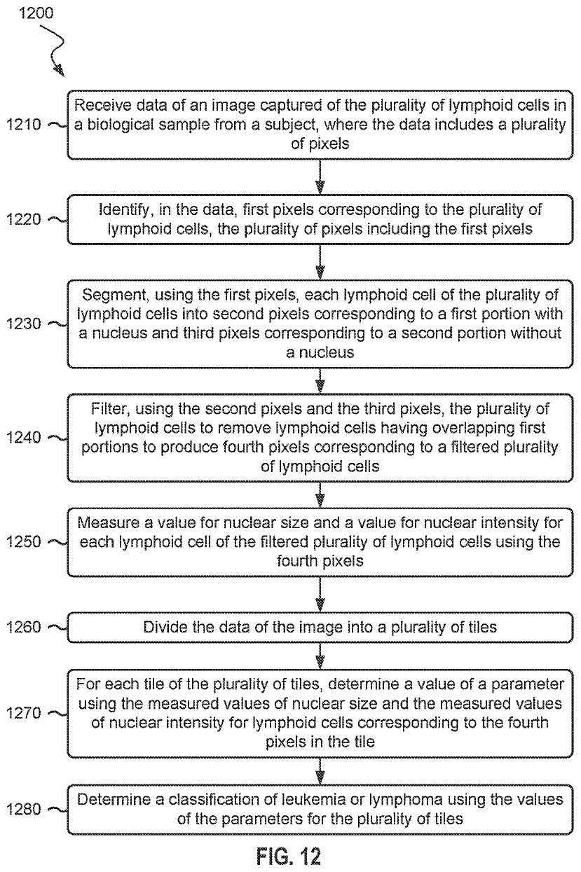

FIG. 12 is a flowchart of an example process 1200 associated with methods and systems for determining leukemia or lymphoma levels using lymphoid images. In some implementations, one or more process blocks of FIG. 12 may be performed by a system (e.g., system 2500). In some implementations, one or more process blocks of FIG. 12 may be performed by another device or a group of devices separate from or including the system. Additionally, or alternatively, one or more process blocks of FIG. 12 may be performed by one or more components of system 2500, such as imaging device 2510, detector 2520, logic system 2530, processor 2550, memory 2535, external memory 2540, storage device 2545, and/or treatment device 2560.

At block 1210, data of an image captured of the plurality of lymphoid cells in a biological sample from a subject is received. The data includes pixels. Block 1210 may be performed in the same or similar manner as described with block 710.

At block 1220, first pixels in the data corresponding to the plurality of lymphoid cells are identified. Block 1120 may be performed in the same or similar manner as described with block 720.

At block 1230, each lymphoid cell of the plurality of lymphoid cells is segmented, using the first pixels, into second pixels corresponding to a first portion with a nucleus and third pixels corresponding to a second portion without a nucleus. Block 1230 may be performed in the same or similar manner as described with block 730.

At block 1240, the plurality of lymphoid cells is filtered, using the second pixels and the third pixels, to remove lymphoid cells having overlapping first portions to produce fourth pixels corresponding to a filtered plurality of lymphoid cells. Block 1240 may be performed in the same or similar manner as described with block 740. In some embodiments, filtering the plurality of lymphoid cells may be optional and the plurality of lymphoid cells may be used rather than the filtered plurality of lymphoid cells.

At block 1250, a value for nuclear size and a value for nuclear intensity is measured for each lymphoid cell of the filtered plurality of lymphoid cells using the fourth pixels. In some embodiments, blocks 1220, 1230, and 1240 may be optional. Measuring the value for nuclear size and the value for nuclear intensity may be performed as described with block 750.

At block 1260, the data of the image may be divided into a plurality of tiles. Block 1260 may be performed in the same or similar manner as described with block 1160.

At block 1270, for each tile of the plurality of tiles, a value of a parameter is determined using the measured values of nuclear size and the measured values of nuclear intensity for lymphoid cells corresponding to the fourth pixels in the tile. Block 1270 may be performed in the same or similar manner as described with block 1170.

At block 1280, a classification of leukemia or lymphoma may be determined using the values of the parameters for the plurality of tiles. The classification may be any classification described herein, including the level of chronic lymphocytic leukemia (CLL), accelerated CLL, or Richter transformation (RT). In embodiments, determining the classification of leukemia or lymphoma in the subject may include determining a statistical value of the values of the parameter for the plurality of tiles. The statistical value may be compared to a cutoff value. In some embodiments, the statistical value may be a mean, median, mode, or percentile of the values. In some embodiments, the statistical value may characterize the type of distribution. The statistical value may indicate how Gaussian, uniform, or shifted the distribution is. A more Gaussian distribution may indicate CLL. A more uniform or smeared distribution may indicate aCLL. A right-shifted distribution may indicate RT.

The cutoff value may be any cutoff value described with process 1100. In some embodiments, however, the cutoff value may be determined for reference values for the plurality of tiles rather than only a subset of the plurality of tiles.

The classification in process 1200 may not include identifying the subset of the plurality of tiles (e.g., proliferation centers). Process 1200 may include values of the parameter determined for all tiles of region of interest.

Process 1200 may include additional implementations, such as any single implementation or any combination of implementations described herein and/or in connection with one or more other processes described herein.

In some embodiments, a machine learning model may be used to determine the classification of leukemia or lymphoma. The plurality of lymphoid cells is a first plurality of lymphoid cells. The value of the parameter is a first value of the parameter. The plurality of tiles is a first plurality of tiles. The machine learning model may be trained by receiving a plurality of training images. Each training image may include a second plurality of lymphoid cells. Each training image of the plurality of training images may be labeled with a known classification of leukemia or lymphoma (e.g., CLL, aCLL, or RT). For each training image of the plurality of training images, the training image may be divided into a plurality of second tiles. A second value of the parameter may be determined for each tile of the plurality of second tiles. The parameters of the machine learning model may be optimized based on outputs of the machine learning model matching or not matching the known classification when the second values for each training image are input into the machine learning model. An output of the machine learning model may specify a classification of leukemia or lymphoma for the training image.

In some embodiments, optimizing the parameters of the machine learning model may include inputting location values corresponding to the second values into the machine learning model. The machine learning model may be any machine learning model described herein, including the ones described with process 700.

The classification of leukemia or lymphoma in the subject may be used as described with process 700. The classification may be compared to classifications at other times. A treatment may be determined and administered based on the classification or the change in classification.

Although FIG. 12 shows example blocks of process 1200, in some implementations, process 1200 may include additional blocks, fewer blocks, different blocks, or differently arranged blocks than those depicted in FIG. 12. Additionally, or alternatively, two or more of the blocks of process 1200 may be performed in parallel.

### c) Classification Using Heat Values of Cells

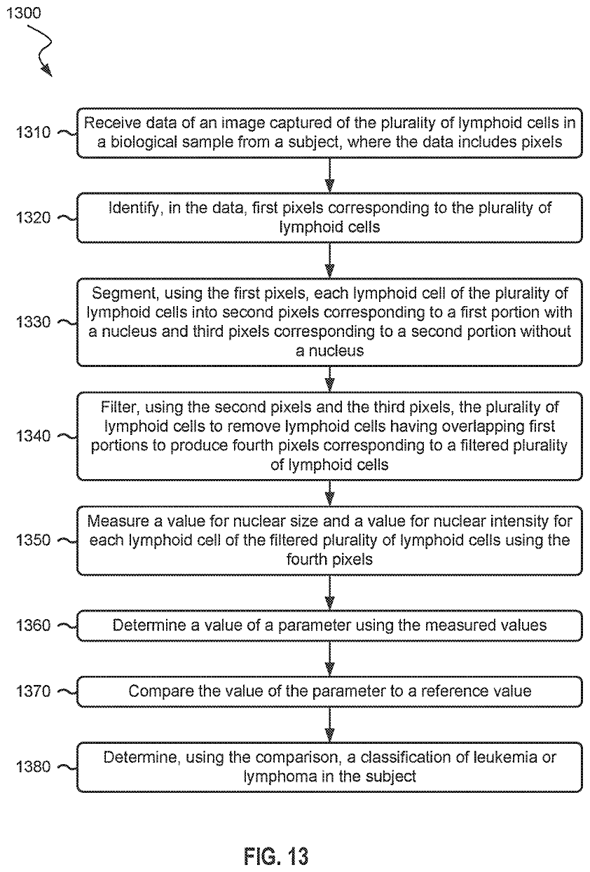

FIG. 13 is a flowchart of an example process 1300 associated with methods and systems for determining leukemia or lymphoma levels using lymphoid images. In some implementations, one or more process blocks of FIG. 13 may be performed by a system (e.g., system 2500). In some implementations, one or more process blocks of FIG. 13 may be performed by another device or a group of devices separate from or including the system. Additionally, or alternatively, one or more process blocks of FIG. 13 may be performed by one or more components of system 2500, such as imaging device 2510, detector 2520, logic system 2530, processor 2550, memory 2535, external memory 2540, storage device 2545, and/or treatment device 2560.

At block 1310, data of an image captured of the plurality of lymphoid cells in a biological sample from a subject is received. The data includes pixels. Block 1310 may be performed in the same or similar manner as described with block 710.

At block 1320, first pixels in the data corresponding to the plurality of lymphoid cells are identified. Block 1320 may be performed in the same or similar manner as described with block 720.

At block 1330, each lymphoid cell of the plurality of lymphoid cells is segmented, using the first pixels, into second pixels corresponding to a first portion with a nucleus and third pixels corresponding to a second portion without a nucleus. Block 1330 may be performed in the same or similar manner as described with block 730.

At block 1340, the plurality of lymphoid cells is filtered, using the second pixels and the third pixels, to remove lymphoid cells having overlapping first portions to produce fourth pixels corresponding to a filtered plurality of lymphoid cells. Block 1340 may be performed in the same or similar manner as described with block 740. In some embodiments, filtering the plurality of lymphoid cells may be optional and the plurality of lymphoid cells may be used rather than the filtered plurality of lymphoid cells.

At block 1350, a value for nuclear size and a value for nuclear intensity is measured for each lymphoid cell of the filtered plurality of lymphoid cells using the fourth pixels. In some embodiments, blocks 1320, 1330, and 1340 may be optional. Measuring the value for nuclear size and the value for nuclear intensity may be performed as described with block 750.

At block 1360, a value of a parameter using the measured values is determined. The value of the parameter may be a heat value described herein. The value of the parameter may be determined using the formula:

$$\frac{1}{{N}_{nucleus}} {\sum }_{i = 1}^{{N}_{nucleus}} \sqrt{S ( {nucleus}_{i} ) * {I}_{mean} ( {nucleus}_{i} )}$$

where N_{nucleus} is the number of nuclei, i is an integer, S(nucleusᵢ) is the size of nucleusᵢ, and Iₘₑₐₙ(nucleusᵢ) is the mean nuclear intensity of nucleusᵢ. In some embodiments, a statistical value (e.g., mean, median, mode, percentile) of the nuclear intensity of nucleusᵢ may be used in determining the parameter.

At block 1370, the value of the parameter is compared to a reference value. The reference value may be determined from a plurality of reference samples. For example, the reference value may be determined from reference samples all having the same lymphoma classification (e.g., CLL, aCLL, or RT). The reference value may be a value that divides one classification from another classification. The reference values may be determined as described with column 920 of FIG. 9 and FIG. 10.

At block 1380, a classification of leukemia or lymphoma in the subject is determined using the comparison. The classification may be determined based on the value of the parameter exceeding or not exceeding the reference value. For example, a classification of RT may be determined if the value of the parameter is greater than or equal to the reference value. A classification of CLL may be determined if the parameter is less than or equal to the reference value. A classification of aCLL may be determined if the value for the parameter is between two reference values, which may be determined from reference samples from subjects having aCLL or from at least two sets of reference samples, with each set having a different classification. The classification may be any classification described herein, including a classification described with block 760.

Process 1300 may include additional implementations, such as any single implementation or any combination of implementations described herein and/or in connection with one or more other processes described herein.

Although FIG. 13 shows example blocks of process 1300, in some implementations, process 1300 may include additional blocks, fewer blocks, different blocks, or differently arranged blocks than those depicted in FIG. 13. Additionally, or alternatively, two or more of the blocks of process 1300 may be performed in parallel.

### II. Unsupervised Learning

Supervised learning can use hand-crafted pathomics features, end-to-end deep learning, or their combination. Unsupervised learning can be used as a knowledge discovery approach to finding meaningful intrinsic patterns in data, which can complement supervised learning and enhance biomedical image diagnostic performance. However, few studies have been proposed to explore the potential role of machine learning algorithms in evaluating lymphomas. Moreover, given the rare incidence of disease transformation in low-grade lymphomas (including CLL), there are limited studies to assess the diagnostic value of AI-based tools.

Here, we show that cellular feature engineering, identifying cellular phenotypes via unsupervised clustering, manifests the most robust performance on pathology slides (accuracy=0.925, AUC=0.978) when compared to other techniques, including those that use other features and patch-based convolutional neural network (CNN) feature extraction. We further validate the reproducibility and robustness of the unsupervised feature extraction via stability and repeated splitting analysis, supporting its potential role in assisting diagnosis in CLL patients with evidence of disease progression.

Embodiments of the present invention by using biomarkers are able to accurately and efficiently assess a level of a disease or a progression of a disease. Surprisingly, only three to six biomarkers, determined through unsupervised learning, can be used to generate accurate results. Using only three to six biomarkers permits computational efficiency. Additionally, methods and systems described herein can use small samples, including core needle biopsy samples. Moreover, accurate analysis is achievable through using a small region of a whole slide image rather than most or all of the whole slide image. Assessing the progression of the disease can be accurately performed, even when assessing progression of a disease may be more challenging than simply identifying the existence of a disease.

### A. Lymphoid Images Study

In this study, we apply a data-driven unsupervised method to identify intrinsic cell populations by clustering cells into multiple phenotypes. We identified three phenotypically distinct cell populations, based on which we extracted morphologic and spatial patterns to build a disease progression diagnostic model. We further evaluated feature engineering through unsupervised clustering and compared its performance with other strategies to handle heterogeneous cell populations, including mixed features, supervised features, unsupervised/mixed/supervised feature fusion and selection, as well as convolutional neural network (CNN) feature extraction. Furthermore, we performed stability and repeated splitting analysis to validate the reproducibility and robustness of the proposed cellular feature engineering via unsupervised clustering.

### 1. Region and Cell Image Analysis

Lymphoid image data from subjects having different stages of CLL are analyzed. Regions of interest are selected in the images. In each region of interest, the cells are segmented to identify nuclei. The nuclei are filtered to exclude overlapping nuclei and low quality nuclei.

### a) Patient Cohort and Data Collection

This study was approved by The University of Texas MD Anderson Cancer Center (UTMDACC) Institutional Review Board and conducted in accord with the Declaration of Helsinki. We retrospectively retrieved patients with hematolymphoid diseases clinically evaluated at UTMDACC between Feb. 1, 2009, and Jul. 31, 2021. Inclusion in this study was based on the following criteria: 1) availability of archived glass slides and digital slides; 2) availability of clinical and laboratory data to support the diagnosis in the electronic medical records; 3) lymph node biopsy confirmed diagnosis. In total, 193 biopsy specimens from 135 patients were eligible for the study, including 69 slides from 44 CLL patients, 44 slides from 34 aCLL patients, and 80 slides from 57 RT patients. The microscopic diagnosis was confirmed on all cases by two hematopathologists, with challenging cases were resolved by a third hematopathologist. To note, a sizable subset (45.5%) of histology slides from nearly half (49.6%) of the patients were obtained and stained in other institutions across the United States, and then transferred to UTMDACC, which markedly increased the diversity and heterogeneity of the slides. Glass slides stained with hematoxylin and eosin (H&E) were scanned as described in section I.A.1.a).

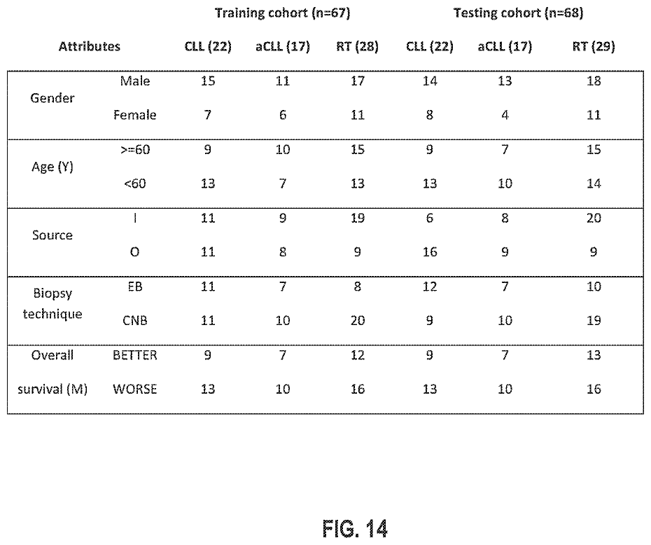

FIG. 14 shows a table of the characteristics of the subjects. The first Ecolumn lists the attribute. The second column lists categories for the attribute. The third through fifth columns are for the training cohort. The sixth through eighth columns are for the testing cohort. The third, fourth, and fifth columns are for the number of subjects with the stage of progression of chronic lymphocytic leukemia for the training cohort. The sixth, seventh, and eighth columns are for the number of subjects with the stage of progression of chronic lymphocytic leukemia for the testing cohort. The “Y” in age represents years. The “M” in overall survival represents months. Source is divided into “I” for in-house case (e.g., UTMDACC) and “O” for outside institution case. EB stands for excisional biopsy. CNB stands for core needle biopsy. Better survival is greater than or equal to 40 months for CLL, greater than or equal to 19 months for aCLL, and greater than or equal to 9 months for RT. Worse survival is less than the months stated for better survival.

### b) Region of Interest (ROI) Selection

Because a regular digital slide of lymph node excisional biopsy with 80,000×50,000 pixels may contain more than two million cells, directly analyzing whole slide images (WSI) on the cellular level is computationally costly. Moreover, the quality of some regions is low and can pose a challenge for automated diagnosis. For example, several tissue artifacts, including folding, crushing, out-of-focus, and section thickness can result in the deterioration of whole-slide tissue assessments. In addition, many regions in the WSI may not be relevant for diagnosis purposes, including areas with red blood cell extravasation, necrosis, and extensive fibrosis. Thus, we adopted the region of interest (ROI) approach to overcome these pre-analytic impediments for cellular feature extraction.

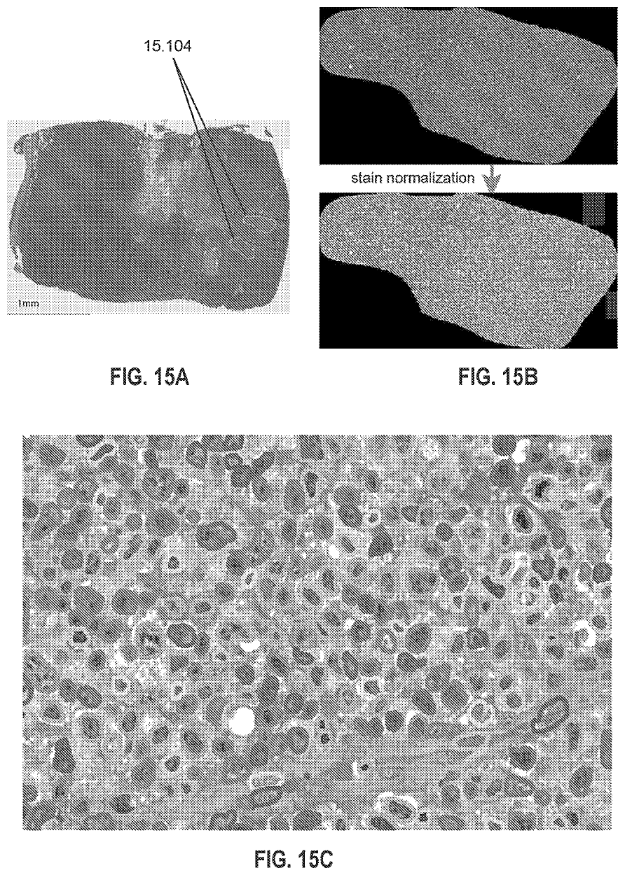

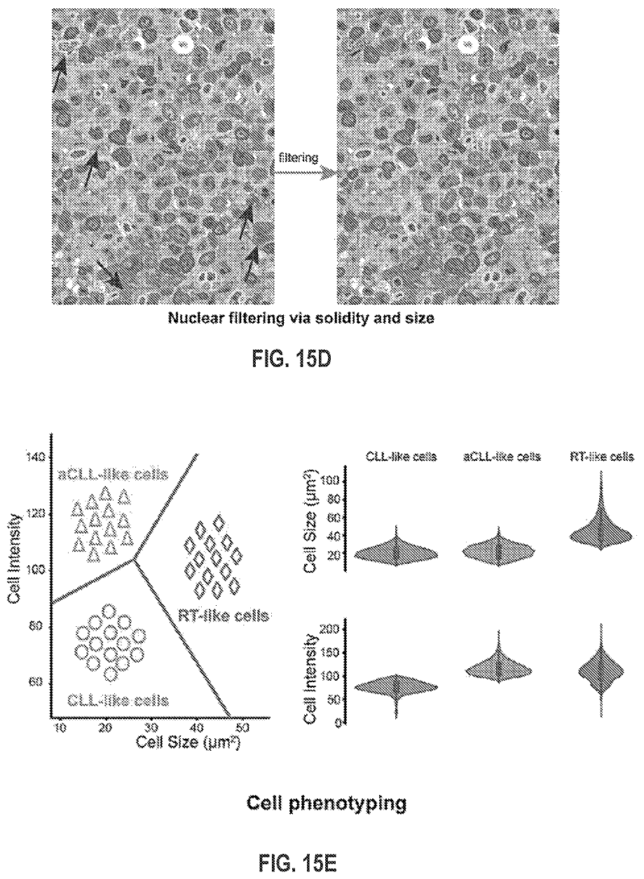

FIG. 15A shows manually selection of diagnostically informative ROIs with high tissue quality for subsequent analysis. Regions 1504 outlined with a green line show the ROIs. The ROIs were selected for regions representative of the tissue and for regions that have high tissue quality. The ROI selection results in a selected region that is representative of the final diagnosis, e.g., if a case was finally diagnosed as RT, the ROI includes the predominant large cells population on the WSI and not the small residual CLL foci in the background. To ensure the inclusion of sufficient cells in each ROI, we set the minimum width and height of the selected ROIs to 512 pixels, which correspond to 0.256 mm in reality. The selected number of ROIs in each slide ranged from 1 to 8. Overall, the number of selected ROIs in CLL, aCLL, and RT was 159, 141, and 165, respectively, and the average ROIs per slide was 2.4.

FIG. 15B shows applying stain normalization in the regions of interest. Reference-free stain normalization with the Vahadane algorithm was performed on all ROIs to mitigate color variations across UTMDACC and outside institutions before subsequent analyses.

### c) Cell Segmentation and Filtering

FIG. 15C shows nuclear segmentation in the region of interest. We performed nuclear segmentation on selected ROIs using the Hover-Net (Graham S V Q, Raza S E A, Azam A, Tsang Y W, Kwak J T, Rajpoot N: Hover-Net: Simultaneous segmentation and classification of nuclei in multi-tissue histology images. Med. Image Anal. 2019, 58:101563). Hover-Net is a deep learning model trained on the PanNuke dataset incorporating 19 different tissue types (Gamper, J. et al., PanNuke: An Open Pan-Cancer Histology Dataset for Nuclei Instance Segmentation and Classification. European Congress on Digital Pathology 2019). Additionally, we performed a quantitative evaluation on 15 manually annotated patches of 256×256 pixels and achieved an overall mean Dice score of 0.825. The Hover-Net had Dice scores of 0.826 and 0.853 on datasets Kumar and CoNSep, respectively (Vu, Q. et al., Methods for Segmentation and Classification of Digital Microscopy Tissue Images. Front Bioeng Biotechnol 2019; Xing, F. et al., Robust Nucleus/Cell Detection and Segmentation in Digital Pathology and Microscopy Images: A Comprehensive Review. IEEE Rev Biomed Eng 2016). Cell segmentation on our dataset was on par with Hover-Net's reported performance. A strong agreement between the manual and Hover-Net segmentations was indicated with a Dice score over 0.80, thus laying the foundation for the proposed cellular-based feature engineering. The Hover-Net results were also visually checked by a hematopathologist to assure nuclear segmentation quality.

FIG. 15D shows using nuclear filtering of solidity and size to exclude overlapping nuclei. Overlapping nuclei post-nuclear segmentation can still pose challenges in downstream analysis, as it may confound nuclear size sensitivity. We used the solidity feature to filter out overlapping segmented cells. By empirically tuning the cutoff with an optimal solidity value of 0.84, we observed that the majority of overlapping nuclei were removed. In addition, we focused on segmented nuclei with pixel numbers between 32 and 432, corresponding to 8 μm² and 108 μm², respectively. This range represented a known biologic nuclear size on histologic slides. Of note, more than 98% of segmented nuclei were retained while nuclei of low quality were removed.

### 2. Pathomics Feature Extraction

Different features of the cells were used to determine the progression of CLL in the subject. Three different categories of features were used. In the first category, the cells were divided into different types through unsupervised clustering. Values of the features for each type were determined and then used to determine the progression of CLL. In the second category, the cells were not divided into different types. Instead, values of features of all these cells that mixed different types were determined. These “mixed” features were used to determine the progression of CLL. In the third category, the cells were divided into small and large cells through supervised learning. Values of features for small cells and large cells were determined and then used to determine the progression of CLL.

### a) Unsupervised Clustering for Cell Phenotyping

Given the prior histologic knowledge and observation of disease progression and transformation including three different lymphoma stages (i.e., CLL progressing to aCLL and finally transforming to RT) it is reasonable to hypothesize that different cell subtypes emerge at different disease stages. We termed the cell subtypes as CLL-like, aCLL-like, and RT-like cells. Based on the three cell subtypes, we designed a data-driven unsupervised clustering strategy to identify three intrinsic cell subtypes based on the appearance of nuclei on WSI (i.e., size and intensity).

FIG. 15E shows characteristics of the different cell subtypes. The graph on the left has cell intensity (nuclear intensity) on the y-axis and cell size on the x-axis. The top graph on the right shows the distribution of cell size among the cell subtypes. The bottom graph on the right shows the distribution of cell intensity among the cell subtypes. The CLL-like cells have a smaller cell size and lower cell intensity than RT-like cells. The CLL-like cells have a lower cell intensity than aCLL-like cells but similar cell size. The aCLL-like cells have a smaller cell size than RT-like cells. The aCLL-like cells have a slightly higher cell intensity than the RT-like cells.

In particular, we employed a spectral clustering algorithm for cell subtype phenotyping. Spectral clustering can provide a robust method to isolate nonconvex and linearly nonseparable clusters, which is ideal for handling heterogeneous cell populations (Filippone, M. et al., A survey of kernel and spectral methods for clustering. Pattern Recogn 2008). However, for patients enrolled in the training set, there were more than 7.5 million cells even after the filtering procedure, rendering it computationally challenging to directly apply our proposed clustering algorithm. To address this issue, we adopted a subsampling scheme, where 5,000 cells were randomly sampled from CLL, aCLL, and RT ROIs, respectively, and then clustering was performed on the pooled cell population. Moreover, we repeated the clustering procedure multiple times with different random sets and varying sample sizes to evaluate cell clustering reproducibility. To propagate the clustering labels to the left out cells, we trained a multiclass logistic regression (LR) model based on cells used for clustering analysis with phenotypes, and then this classifier was applied to label millions of segmented cells into one of the newly discovered phenotypes. In the end, we extracted two types of features, including cell ratio and density, to characterize individual cell phenotypes. Cell ratio considers the proportions of individual cell phenotypes inside an ROI, and cell density measures the compactness of individual cell types in a given ROI.

### b) Comparison to Alternative Ways of Cell Profiling

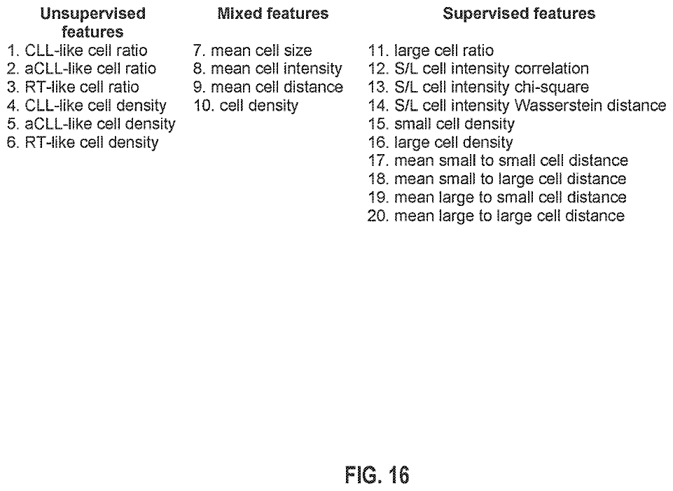

FIG. 16 is a table showing the list of three different sets of features used to classify the tissue sample. The first column lists “Unsupervised features.” The “unsupervised features” include six cellular features that were extracted based on the unsupervised clustering scheme described above. The second column lists “Mixed features,” where no distinction between cell subtypes was made and features based on the “mixed” cells were used. The third column lists “Supervised features,” where cells were split into small and large cells in a supervised manner.

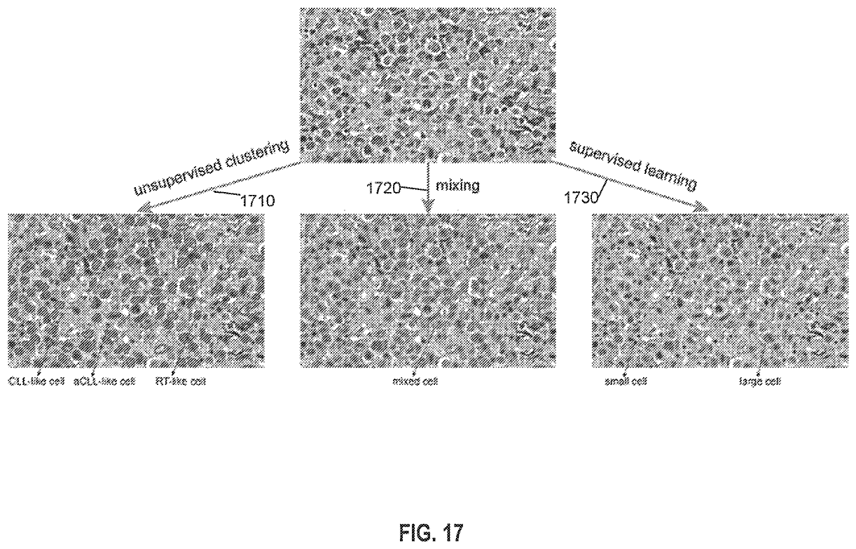

FIG. 17 shows three different ways of analyzing a tissue sample. Technique 1710 involves unsupervised learning and results in the unsupervised features of FIG. 16. Technique 1720 is based on mixed cell populations and results in the mixed features of FIG. 16. Technique 1730 involves supervised learning and results in the supervised features of FIG. 16. In the following, we elaborate on pathomics feature extraction based on mixed cells and supervised learning approaches.

### (1) Feature Extraction Via Mixed Cells

For the mixed cell analysis (technique 1720 in FIG. 17), no intrinsic subtype distinction was made. Instead, we directly mixed cells to prepare them for feature extraction. This technique is described in more detail in Hussein, S. et al., Artificial intelligence strategy integrating morphologic and architectural biomarkers provides robust diagnostic accuracy for disease progression in Chronic Lymphocytic Leukemia. J Pathol 2021. Two characteristic cytologic features (nuclear size and nuclear intensity) and two characteristic spatial distribution patterns (cellular density and cell to nearest-neighbor distance) were extracted for the diagnosis and termed mixed features (as shown in FIG. 16). For nuclear size, nuclear intensity, and cell to nearest-neighbor distance, we measured the value at every single cell and averaged it across all cells inside each ROI. Cellular density was defined as the number of cells inside the ROI divided by the area covered.

### (2) Feature Extraction Via Supervised Learning

From a pathology practice standpoint, nuclear size is a predominant factor in the differentiation of CLL from its accelerated and transformed phases. To capture nuclear size differences, we aimed to define small versus large cell subtypes via a supervised manner (technique 430 in FIG. 17). Based on the training ROIs, we optimized the cutoff value by maximizing the separation margin among CLL, aCLL, and RT. To be specific, by introducing a size cutoff variable, we partitioned all cells into small and large cells in an ROI, where the large cell ratio was defined as the number of large cells over the total number of cells. Then, we calculated the average large cell ratios of three disease subtypes as R_{CLL}, R_{acLL}, and R_{RT} with a given cell size cutoff value. An objective function (R_{RT}−R_{acLL})*(R_{acLL}−R_{CLL})*(R_{RT}−R_{CLL}) was formulated. By optimizing the cutoff value, we maximized the separation among CLL, aCLL, and RT on the training set. By crossing through all possible cell size values from the minimum of 8 μm² to the maximum of 108 μm², we discovered that the value of 24 μm² was the optimal cutoff in this example. Since the ROI's disease label information was leaked during the optimal cutoff searching process, we termed the small and large cell isolation process as supervised learning.

Similar to feature extraction of mixed cells, here we also extracted cellular features based on nuclear size, intensity, density, and distance. For nuclear size, we measured the large cell ratio. For nuclear intensity, we first generated the probability distribution function (PDF) of intensity histogram for both small and large cells inside each ROI, we then measured the similarity between small and large cell PDFs with correlation, Chi-Square, and Wasserstein distance, respectively, which resulted in three intensity-related features. In addition, we calculated both the small and large cell densities. We also measured four types of cell distance features, including the average small cell to its nearest small cell neighbor distance, small to large, large to small, as well as large to nearest large cell neighbor distance. Overall, 10 features were obtained based on cellular phenotyping with supervised learning, which we termed supervised features as shown in FIG. 16. These 10 features were selected by taking into account practitioner experience with diagnosing leukemia while qualitatively comparing small and large cells.

### 3. Machine Learning Models for Disease Progression Prediction

Machine learning models were trained using the unsupervised features, mixed features, and supervised features and then tested. For comparison, a convolutional neural network (CNN) model was trained and tested on the same images.

### a) Cellular Feature-Based Diagnosis Models

We aimed to build prediction models based on pathomics features to investigate their performance for disease progression diagnosis. In total, we extracted 20 features based on the three different manners to phenotype heterogeneous cells, including six from unsupervised clustering, four from mixed cells, and ten from supervised learning as shown in FIG. 16. First, we trained separate prediction models for three feature types as well as the fused pathomics features. We utilized the XGBoost algorithm to perform the diagnosis analysis based upon the extracted features from each ROI (Chen T Q et al., KDD '16: Proceedings of the 22nd ACM SIGKDD International Conference on Knowledge Discovery and Data Mining 2016). XGBoost is an ensemble classifier and implements the gradient boosted decision trees, which is known to work well on small sample datasets and can outperform support-vector machine (SVM) and random forest. In addition, we also conducted feature selection on the extracted 20 features via the classical meta-transformer algorithm, which employed a tree-based estimator to compute the impurity-based feature importance (Deng, H T et al., Feature selection via regularized trees, IEEE IJCNN 2012). XGBoost version 1.4.1 was used (github.com/dmlc/xgboost). The top-ranked features were retained to train an XGBoost model, where we set the threshold at 0.02 to filter out less relevant features. In the end, all of the trained models were independently validated using the testing cohort.

Furthermore, we evaluated the robustness of the models through repeated splitting analysis, where we randomly split the whole dataset into training and testing cohorts 100 times, stratified at patient level with a ratio of 1:1. Patient-based splitting was used to avoid selecting ROIs belonging to the same patients in both the training and testing sets, which can lead to information leakage and model overfitting. The XGBoost models were trained specifically on an individual training set and validated on the testing set. We computed the mean and standard deviation of disease diagnosis performance metrics on the 100 times splitting-basis to mitigate any potential biases that might be caused by one-time splitting, which further validated our model's effectiveness.

### b) Comparison to the Convolutional Neural Network (CNN)

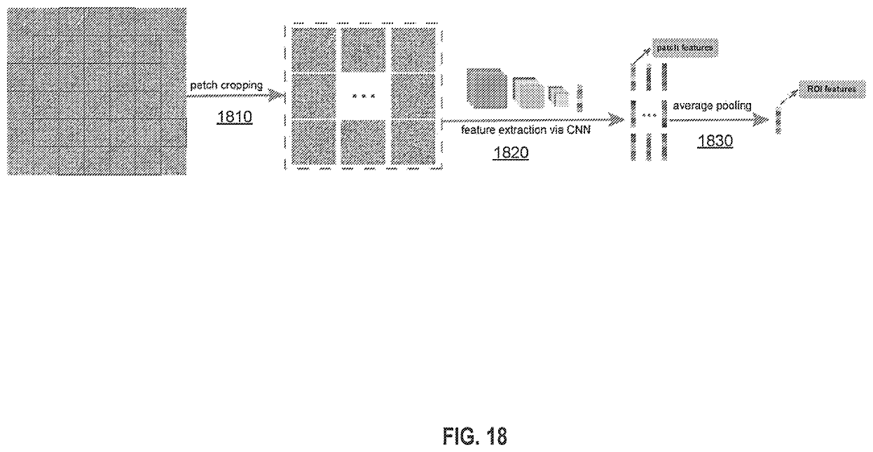

For comparison purposes, we also employed the patch-based CNN model, which is the most common way for digital slide image analysis since the renaissance of deep learning. FIG. 18 illustrates the procedures of using CNN to extract ROI features. At stage 1810, we first divided an ROI into non-overlapping patches with 512×512 pixels and discarded those patches with less than 90% pixels inside the ROI. We adopted the transfer learning scheme to fine-tune the ResNet-50 model, which was pre-trained on the ImageNet, on the cropped patches from the training patient cohort. The early stopping mechanism was used to choose the best patch classifier when the training loss began to plateau. At stage 1820, with the fine-tuned CNN model, we took the output of the penultimate layer with a dimension of 2048, to extract the patch-wise features. At stage 1830, because an ROI could contain multiple patches, we performed the average pooling on all of its patches' features. After the patch-based CNN feature extraction, each ROI was represented as a 2048 feature vector and termed as CNN ROI features. To be consistent, we performed the diagnosis on these ROI features with the XGBoost classifier.

### 4. Results

The accuracy (ACC) and macro-average of the receiver operating characteristic (ROC) area under the curve (AUC) were measured to assess the performance of the disease diagnostic models. The one-tailed t-test (upper tail) was employed to measure if the diagnosis performance of one model is statistically better than the other.

### a) Discovery and Validation of Three Cellular Subtypes

We identified three cell phenotypes after spectral clustering, where CLL-like cells with small nuclear size and low nuclear intensity, aCLL-like cells with small nuclear size but higher nuclear intensity, and the RT-like cells with relatively large nuclear size. The multiclass logistic regression (LR) model was fitted with the clustered pseudo-labels and the decision boundaries were obtained to label the rest of the cells.

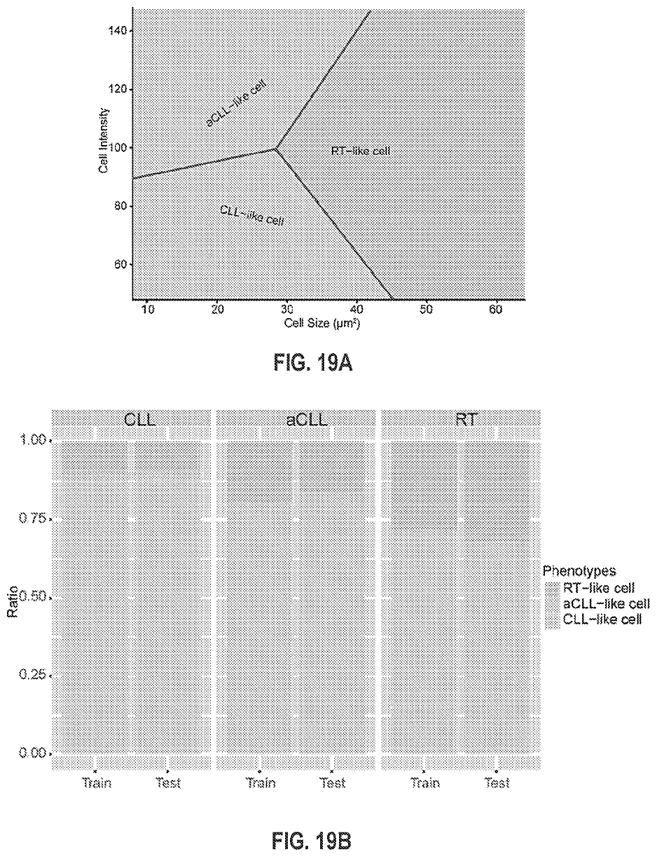

FIG. 19A shows the decision boundaries between the three subtypes. The x-axis shows the cell size in square microns. The y-axis shows the cell intensity. The graph is similar to FIG. 2E with the cell subtypes in a similar arrangement.

FIG. 19B shows the distribution of cell subtypes for the different stages of disease. The x-axis shows the training and testing cohorts for different stages of chronic lymphocytic leukemia disease. The y-axis shows the ratio of each cell subtype. The top portion (red) shows the RT-like cell amount. The middle portion (blue) shows the aCLL-like cell amount. The bottom portion (green) shows the CLL-like cell amount. We found that the ratios of these three types of clustered cells were quite consistent in training and testing sets across CLL, aCLL, and RT, with the ratio of RT-like cells was gradually increased from CLL to aCLL, and RT, while the ratio of CLL-like cells showed an opposite trend. These results demonstrated cellular phenotype evolution in tandem with CLL progression to RT and confirmed the hypothesis that different cell subtypes enriched at different disease stages.

### b) Clustering-Based Model Obtains the Best Performance for Disease Progression Prediction

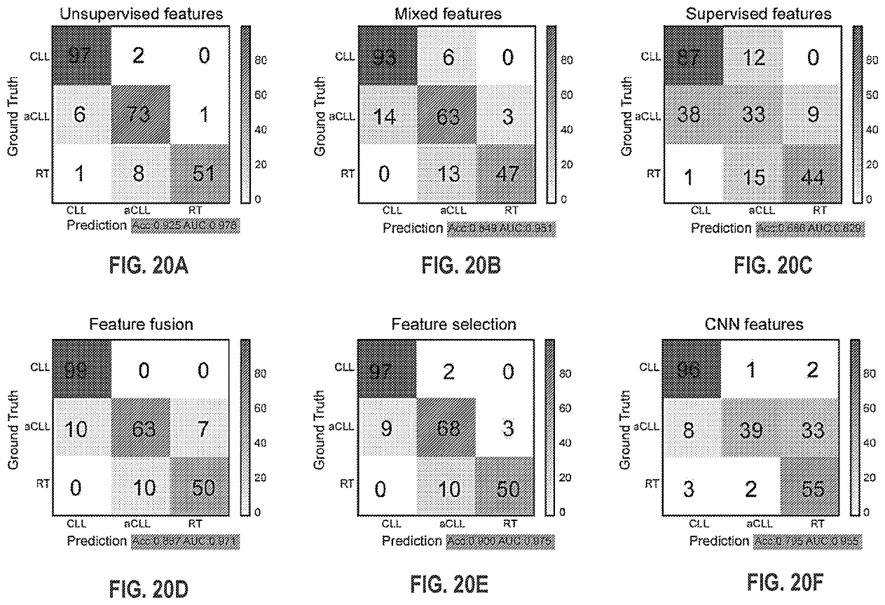

FIGS. 20A-20G show accuracy and AUC results of analyzing disease progression using features generated from different techniques. The y-axis shows the ground truth stage of disease progression. The x-axis shows the predicted stage of disease progression. The number represents the number of samples corresponding to the ground truth and the prediction. The accuracy and the area under the curve are shown underneath each figure.

We trained the XGBoost classifier on the ROIs from patients in the training set and evaluated the diagnostic performance in the testing set. Of the three models in FIGS. 3 and 4 using pathomic features, the model built on unsupervised features achieved the best diagnostic performance with an accuracy of 0.925 and AUC of 0.978, as shown in FIG. 20A. By contrast, the mixed features model attained a lower accuracy of 0.849 as shown in FIG. 20B. The supervised features model had the lowest performance (accuracy=0.686, AUC=0.829) as shown in FIG. 20C.

Next, we combined these three feature types to build a composite model, which attained a modest performance (accuracy=0.887, AUC=0.971) between models based on all 20 features listed in FIG. 16. FIG. 20D shows results using all 20 features.

FIG. 20E is based on a selection of 13 highly ranked features. The selection of the features is described below. The model built on these selected features obtained an accuracy of 0.900 and AUC of 0.975, outperforming the model that directly trained on fused features in FIG. 20D. This indicates that removing uninformative features can boost a model's diagnostic performance.

FIG. 20F is based on features determined from a CNN model. Surprisingly, the CNN model had moderate performance with an accuracy of 0.795, where a considerable amount of aCLL patients were misclassified as RT during testing. The CNN model had an AUC of 0.955, which is still lower than the unsupervised features and other models.

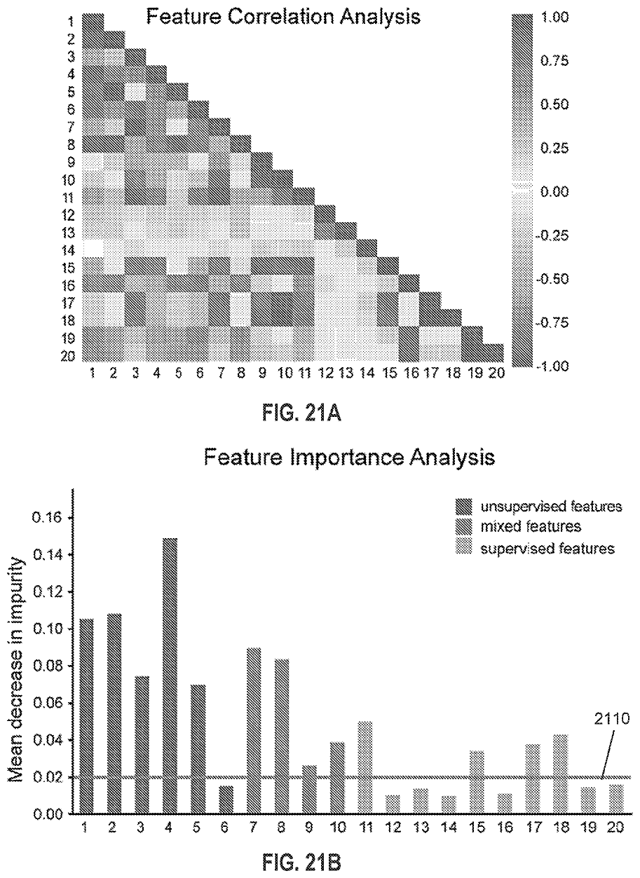

FIG. 21A shows a feature correlation analysis of the 20 features from the unsupervised features, mixed features, and supervised features. The feature correlation provides information about how two features correlate with each other, e.g., how changes in one feature tend to occur with a corresponding change (or inverse change for negative values) in samples. This correlation information could be used to reduce the number of features, e.g., by a dimensional reduction procedures, such as principal component analysis.

The x-axis and y-axis show the number of the feature, corresponding to the numbered features in FIG. 16. A redder color indicates a higher positive correlation. A bluer color indicates a higher negative correlation. A yellower color indicates lower correlation. The feature correlation heatmap of these 20 features showed some high and low correlations among certain feature pairs. For example, the correlation coefficient between the RT-like cell ratio in unsupervised features (feature 3) and the mean cell size in mixed features (feature 7) was 0.939. The redundancy among the fused features as well as some less informative features might have caused this intermediate performance.

FIG. 21B shows the importance of individual features. The x-axis lists the feature number, corresponding to the numbers in FIG. 16. The y-axis shows the mean decrease in impurity. The mean decrease in impurity importance metric refers to the improvement in the “Gini gain” splitting criterion. The metric incorporates a weighted mean of the individual improvement in the splitting criterion produced by each variable. A higher mean decrease in impurity indicates a more important feature. We applied a feature selection algorithm, which selected 13 features with highly ranked information, including five features from clustering, four from mixed cells, and four from supervised learning. The 13 features are above horizontal line 2110. Interestingly, the top three important features are from the unsupervised feature set, including the CLL-like cell density, CLL-like cell ratio, and aCLL-like cell ratio. By contrast, features from the supervised set are generally associated with lower importance.

### c) Clustering-Based Model Shows High Reproducibility and Robustness

We tested the stochastic effect of a random selection of cells from CLL, aCLL, and RT ROIs on the unsupervised clustering procedure to ensure reproducibility. We reported the results of eight experiments of different random cell selections.

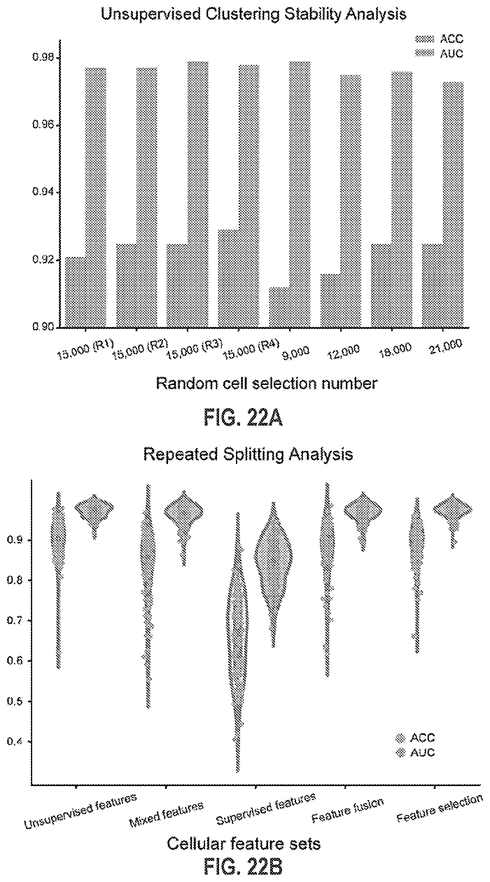

FIG. 22A shows the results of unsupervised clustering stability analysis. The x-axis shows the number of cells randomly selected and has columns representing accuracy and AUC. The y-axis shows the accuracy or AUC. In the first four studies, where 15,000 cells were randomly selected but with different random states to ensure the stochasticity in the selection, we observed close performance, with accuracy ranging from 0.921 to 0.929 and AUC ranging from 0.977 to 0.979. Next, by changing the number of selected cells, we observed a notable accuracy improvement when size increased from 9,000 (accuracy=0.912) to 12,000 (accuracy=0.916) compared with the employed 15,000 (accuracy=0.925). Then, the performance plateaued when we further increased size to 18,000 (accuracy=0.925) and 21,000 (accuracy=0.925).

Next, we conducted the repeated training and testing splitting analysis to validate the general performance of the proposed clustering-based cellular feature extraction strategy and others. Here, we randomly split the patients into training and testing cohorts 100 times with a ratio of 1:1, and repeated the diagnostic model construction and validation, where the performance including the accuracy and AUC.

FIG. 22B shows results from repeated splitting analysis. The x-axis shows the different models with columns representing accuracy and AUC. The y-axis shows the accuracy or AUC. The width of the data plotted shows the distribution of the results. Similar to the single splitting analysis, the proposed unsupervised clustering manner obtained the best mean accuracy and AUC, with the values of 0.902 and 0.973, respectively. The mean accuracies of mixed, supervised, fusion, and selection feature sets were 0.835, 0.662, 0.876, and 0.891, and the mean AUC values of these four compared manners were 0.961, 0.841, 0.969, and 0.973. Among them, feature selection exhibited the closest outcome with the unsupervised features. Nevertheless, we conducted the one-tailed t-test between the unsupervised model and feature selection accuracies and acquired a p-value of 0.043, which demonstrated the superiority of the proposed unsupervised cellular feature analysis. Taken together, these results demonstrate the superiority and robustness of the proposed unsupervised cellular feature engineering. We observed that the mean accuracies of the repeated splitting analysis are generally 1-3% lower compared with the previous training and testing analysis with balanced splitting, suggesting that the unbalance splitting of gender, age, etc. between training and testing cohorts can negatively affect the performance.

### d) Ablation Study to Understand Feature Importance

Subsets of the six unsupervised features (CLL-like cell ratio, aCLL-like cell ratio, RT-like cell ratio, CLL-like cell density, aCLL-like cell density, RT-like cell density) were tested in an ablation study to understand the importance of different features. In the experiments, using all six features achieved a mean accuracy of 0.902. The statistical significances of differences in accuracies of subsets of six features from using all six features was determined.

When removing one of the features (using five features), all subsets show a lower mean accuracy. However, the lower mean accuracy is not significantly different from using all six features, with the exception of the subset that does not use CLL-like cell density. These results suggest that CLL-like cell density may be the most important feature.

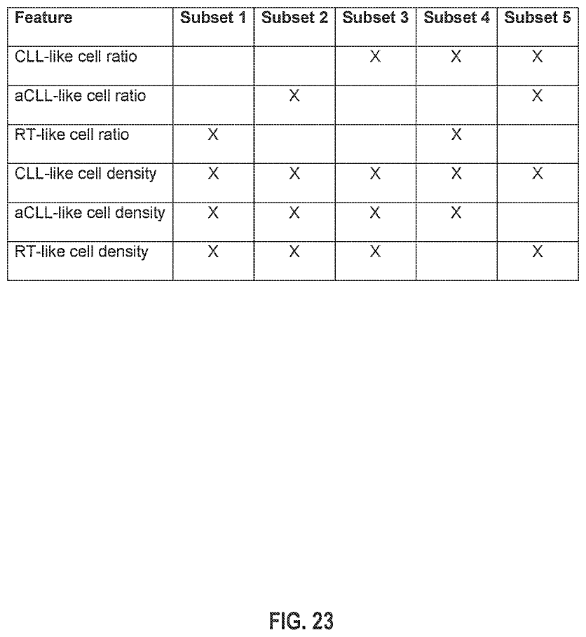

FIG. 23 shows a table of results from removing two of the features (using four features). The rows show the six features. The columns show the subsets showing no significant difference in accuracy from using six features. An “X” in a table When removing two of the features (using four features), five subsets do not show a significant drop. The five subsets all include CLL-like cell density.

When removing three features (using three features), only one subset does not show a significant drop. The subset uses CLL-like cell density, aCLL-like cell density, RT-like cell density. The mean accuracy was 0.894. The result suggests that the cell density may be a more important feature than cell ratio.

When removing four features (using two features), all combinations show significant drops in accuracy compared with using all six features.

### 5. Discussion

In this study, we hypothesized that phenotyping cells in the CLL and its progressed phases and then extracting features based on fine-grained cellular phenotypes can enhance the CLL diagnostic performance. Subsequently, we discovered and validated three cellular phenotypes with unsupervised clustering, and these cellular subtypes demonstrated the distinct size and intensity features, corroborating with clinical observations of disease transformation in CLL patients. Further, our trained model, based on six pathomics features characterizing these three cellular subtypes, achieved the highest performance for diagnosing the three disease entities. By contrast, the alternative ways to extract pathomics features, including mixed and supervised schemes, were associated with lower diagnostic accuracy. Interestingly, integrated analysis of different types of pathomics features failed to improve prediction accuracy, indicating the non-synergistic interactions among different pathomics feature types. In addition, our proposed unsupervised clustering-based model has shown superior performance compared to the state-of-the-art deep learning approach.

Data suggests that the prognosis of aCLL patients is poorer than that of CLL patients and switching into a more intensive treatment regimen in patients with aCLL is deployed with better clinical response, especially in CLL patients who become refractory to treatment. Thus, distinguishing classic CLL from its accelerated phase morphologically is important. Determining aCLL can confirm clinical suspicion of disease progression and support a decision to upgrade treatment. In clinical practice, the distinction between CLL and aCLL in patients being evaluated for progressive disease can be challenging, particularly in small needle-biopsy specimens, the most commonly sought-after biopsy technique for diagnostic confirmation in the setting of clinical suspicion of disease progression. CLL may harbor progressive phases that can be challenging to capture by standard morphology assessment alone and may benefit from the implementation of extra-resources to enhance diagnostic accuracy. We envision that the newly proposed machine learning models can potentially help fill the clinical gap by providing an immediate and accurate diagnosis with no or little additional cost to subjects.

The unsupervised clustering techniques described herein are among the first to have an unsupervised learning model enhance the diagnostic accuracy of CLL progression. Interestingly, three phenotypically distinct cellular populations were identified and validated, and their relative composition changes during disease progression. Further, by taking advantage of both the cytologic and spatial cellular features of these newly phenotyped cells, the proposed cellular feature engineering model obtained the best performance among all compared methods, including both conventional pathomics and deep learning methods.

The superiority of the unsupervised cellular features can also be inferred from feature importance analysis. As shown in FIG. 21B, five of six unsupervised features are ranked with high importance, and the least ranked important of these five features was even greater than the supervised features with the highest importance. Although all four mixed features were retained after feature selection, the values of their importance were generally inferior to the five selected unsupervised features. While these three manners of cell characterization can be seen as profiling tumor microenvironment from different perspectives, direct feature fusion results in significant deterioration of model performance, possibly due to the pollution from irrelevant and redundant information from supervised features. This highlights the importance of extracting meaningful pathomics features, which may not follow the rule of the more the better.

Convolutional Neural Networks (CNNs) are the state-of-the-art in most fundamental image recognition tasks, including image classification, object detection, semantic segmentation. The main concern in this patch-based manner is the lack of clinical relatability of the extracted CNN deep features, which blends different cell types and their background context through a cascade of the convolutional and pooling operations, similar to classical bulk sequencing. In this study, we took the advantage of the outstanding representation learning power of the CNN algorithm to segment massive nuclei from WSIs. Then, we focused on key components (i.e., different cell types) of high diagnostic values from the heterogeneous histologic landscape and extracted pathomics features accordingly. Different from the patch-based CNN approach, our method can be viewed as a step toward single cell sequencing. Moreover, we emphasized on the interpretability of these cellular features, rather than the “black-box” scheme of classical CNN method. Consequently, the diagnostic performance of our proposed model highlighted many advantages of the cellular-based features over the CNN extracted features, indicating that a patch-based CNN may not be able to capture the subtle cellular transformation during disease progression. Especially, given that aCLL is the intermediate phase between CLL and RT and exhibits overlapping morphologic characteristics of both CLL and RT, CNN model misclassified a large amount of aCLL patients into RT. This may be caused by 1) a sample of CNN only covers a limited area (512×512 pixels), which might hinder its ability to differentiate the three disease entities, and 2) CNN model may not be well trained with around 100 patient samples rather than millions of new samples in computer vision field since RT is a rare disease. By contrast, the proposed cellular features consider all cells in the given ROI, this strategy has demonstrated some advantages by covering a larger tissue field.

In conclusion, we propose a novel cellular feature engineering via unsupervised clustering to diagnose CLL, aCLL, and RT based on WSIs. Extensive experiments show that the proposed unsupervised features model reveals superiority over mixed features, supervised features, unsupervised/mixed/supervised feature fusion and selection, and CNN features. This study demonstrates the potential of applying an unsupervised clustering approach to enhance the diagnostic accuracy of CLL in its progressive phases.

### B. Example Methods

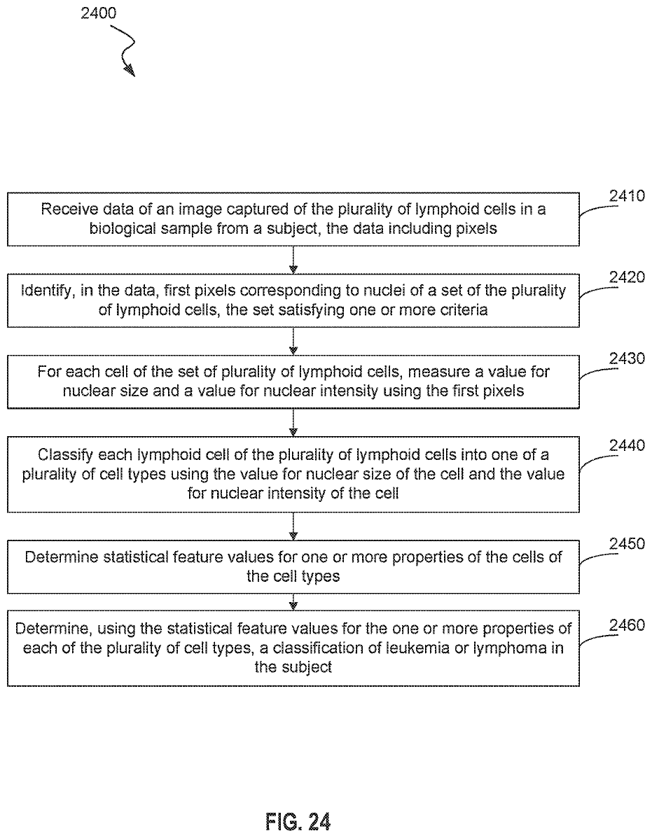

FIG. 24 is a flowchart of an example process 2400 for analyzing a plurality of lymphoid cells. Additionally, or alternatively, one or more process blocks of FIG. 24 may be performed by one or more components of system 2500, such as imaging device 2510, detector 2520, logic system 2530, processor 2550, memory 2535, external memory 2540, storage device 2545, and/or treatment device 2560.

At block 2410, data of an image captured of the plurality of lymphoid cells in a biological sample from a subject is received. The lymphoid cells may be treated with a stain. The stain may include hematoxylin and eosin. The data includes pixels. The data of the image may include at least 100,000 pixels, including, for example, 100,000 to 250,000, 250,000 to 500,000, 500,000 to 1 million, 1 to 2 million, 2 to 5 million, 5 to 10 million, or more than 10 million pixels. The pixels may be two-dimensional. In some embodiments, the data may include three-dimensional pixels (i.e., voxels). The data may include values for color intensities of each pixel. The color intensities may be for each color channel of a plurality of color channels. For example, the plurality of color channels may be RGB or another color triplet. In some embodiments, the data may include values for grayscale intensity. The data of each pixel may include coordinates of the pixel relative to the image. In some embodiments, the data of each pixel may include coordinates relative to a location within a slide or within a tissue.

Process 2400 may include obtaining the biological sample from the subject. The biological sample may be obtained as a biopsy. The biopsy may be a needle core biopsy or an excisional biopsy. The biological sample may be from a lymph node. Process 2400 may include capturing the image of the plurality of lymphoid cells in the biological sample by performing microscopy on the biological sample. Microscopy may be by digital microscopy, e.g., Aperio AT2 brightfield scanner.

The plurality of lymphoid cells may be in a region or regions of interest of the image. The image may be a whole slide image. The plurality of lymphoid cells may be a first plurality of lymphoid cells. The image may include a second plurality of lymphoid cells. Process 2400 may further include selecting a region that includes the first plurality of lymphoid cells. The region or regions may be selected as described herein. The region may be selected as representative of the biological sample from the subject. The region may be selected to include the predominant shape or size of cell in the image. The region may be selected for an area that appears to have lymphoma or leukemia, using a pathologist's judgment. The image may be divided into a number of regions (e.g., a grid), and an image quality score may be evaluated for each region. The image quality score may be determined using the color intensity, sharpness, contrast, and/or other image characteristics of the region. Additionally, the image quality score may also include a determination of the relevance of the particular tissue location to the classification of leukemia or lymphoma levels. The region of interest may be selected from regions having a minimum image quality score. In some embodiments, the selection of the region of interest may be by a hemapathologist. In other embodiments, the selection of the region of interest may be by a computer. The region may have a minimum width and height of 500, 600, 700, 800, 900, 1,000, 1,500, or 2,000 pixels. The number of selected regions may be from 10 to 50, 50 to 100, 100 to 150, 150 to 200, 200 to 300, 300 to 500, or more than 500. The region(s) may be selected prior to block 2420.

In some embodiments, the data may be normalized. For example, the data may be normalized based on a maximum intensity value. The data may include an intensity value for each color channel. Each color channel may be normalized based on one or more maximum intensity values. In some embodiments, pixels with outlier intensity values may be replaced with the maximum intensity value.

At block 2420, first pixels corresponding to nuclei of a set of the plurality of lymphoid cells are identified in the data. The identification of the plurality of lymphoid cells may include analyzing color intensity of pixels in the data. Pixels with a color intensity matching or within a certain range of the expected color intensity of a stained cell may be identified as corresponding to a lymphoid cell. Continuous areas of pixels with the requisite color intensity may be grouped together as a single cell.

The set may satisfy one or more criteria. Process 2400 may include filtering the plurality of lymphoid cells by the one or more criteria to obtain the set. The one or more criteria may include a threshold nuclear size value, a threshold nuclear intensity value, a threshold value for area to perimeter, a threshold solidity value, a threshold major axis length, a threshold eccentricity, or a threshold roundness. The threshold value may be a minimum or a maximum cutoff. The threshold value may be normalized to vales for the cell or portions of the cell without the nucleus. For example, the nuclear size value may be a ratio of the size of the nucleus to the size of the cell without the nucleus. In some embodiments, filtering may include determining the size of the nuclei. The filtered cells may include only cells with nuclei in a certain size range. For example, the nuclei may be represented by pixels corresponding to sizes from 8 μm² to 108 μm², 5 μm² to 10 μm², 10 μm² to 50 μm², 50 μm² to 80 μm², 80 μm² to 100 μm², or 100 μm² to 110 μm². The threshold value may be set by determining a value corresponding to a certain percentile of a distribution or a certain number of standard deviations (e.g., 2 or 3) away from the mean of a distribution.

The set of the plurality of lymphoid cells may include 1,000 to 2,000, 2,000 to 5,000, 5,000 to 10,000, 10,000 to 15,000, 15,000 to 20,000 or over 20,000 lymphoid cells. The plurality of lymphoid cells may have at least 1%, 2%, 5%, 10%, 20%, 30%, 40%, 50%, 60%, 70%, 80%, 90%, or 100% more cells than the set of the plurality of lymphoid cells.

Identifying the first pixels may include nuclear segmentation. Identifying the first pixels corresponding to the nuclei of set of the plurality of lymphoid cells may include identifying, in the data, second pixels corresponding to the plurality of lymphoid cells. Each lymphoid cell of the plurality of lymphoid cells may be segmented, using the second pixels, into third pixels corresponding to a first portion with a nucleus and fourth pixels corresponding to a second portion without a nucleus. The second portion may be the cytoplasm portion of the cell. The segmentation may involve determining that intensities the third pixels are closer to a reference intensity for stained nuclei than a reference intensity for cytoplasm of the cell. In some embodiments, the segmentation may involve analyzing contrast between neighboring pixels, where areas of high contrast indicate a boundary between a nucleus and the cytoplasm. Segmentation may be by a machine learning model, including a convolutional neural network. Segmentation may be performed using the Hover-Net model. The plurality of lymphoid cells may be filtered, using the third pixels and the fourth pixels, to remove lymphoid cells having overlapping first portions to produce the first pixels.

At block 2430, a value for nuclear size and a value for nuclear intensity are measured using the first pixels for each cell of the set of the plurality of lymphoid cells. The values may be measured from data of an image captured of the plurality of lymphoid cells. Pixels corresponding to nuclei of the plurality of lymphoid cells may be identified. These pixels may then be used for measurements.

Measuring the value for nuclear intensity may include measuring subvalues for a plurality of color channels and determining a statistical value of the subvalues for the plurality of color channels. For example, a first subvalue may be measured for a red channel, a second subvalue may be measured for a green channel, and a third subvalue may be measured for a blue channel. Each subvalue may be a statistical value of all values measured for the filtered plurality of lymphoid cells. For example, the first subvalue for the red channel may be a mean, median, mode, or percentile of intensity values. A statistical value of the subvalues for the plurality of color channels may be determined. The statistical value may be a mean or median of the subvalues for the plurality of color channels. In other embodiments, the value for nuclear intensity may include measuring intensity based on grayscale (either white or black represent maximum intensity).

At block 2440, each lymphoid cell of the plurality of lymphoid cells is classified into one of a plurality of cell types using the value for nuclear size of the cell and the value for nuclear intensity of the cell. FIG. 2E shows an example clustering of the cells into three types using cell size and cell intensity, which are determined by nuclear size and nuclear intensity, respectively. Classifying each lymphoid cell of the plurality of lymphoid cells may include unsupervised clustering performed by a computer system, such as logic system 1130.

A variety of clustering techniques can be used, such as spectral clustering, any clustering technique described herein, or other techniques known to the skilled person. Other example clustering techniques can include k-means, density-based spatial clustering of applications with noise (DBSCAN), hierarchical clustering, mixture models, biclustering, and ordering points to identify the clustering structure (OPTICS). As further examples, the clustering can be connectivity-based, centroid-based, distribution-based (such as such as multivariate normal distributions used by the expectation-maximization algorithm), or grid-based.

The clustering can be performed directly on the measured data points or on a dimensionally-reduced set of data point. For example, clustering may include performing principal component analysis (PCA) or other dimensionality-reduction methods, such as diffusion maps, or by using force-based methods such as t-distributed stochastic neighbor embedding (t-SNE).

The plurality of cell types may include chronic lymphocytic leukemia-like (CLL-like) cells, accelerated CLL-like (aCLL-like) cells, and Richter transformation-like (RT-like) cells. In some embodiments, the plurality of cell types may include additional types. For example, cell types may include different subcategories of each type. In some embodiments, the cell types may not correspond to a severity of a disease, and the clustering may be based on groups formed with cell size and cell intensities.

The CLL-like cells may be characterized by a first statistical nuclear size value for and a first statistical nuclear intensity value. The aCLL-like cells may be characterized by a second statistical nuclear size value and a second statistical nuclear intensity value. The RT-like cells may be characterized by a third statistical nuclear size value and a third statistical nuclear intensity value. The first statistical nuclear size value may be less than the third statistical nuclear size value. The first statistical nuclear intensity value may be less than the second statistical nuclear intensity value and the third statistical nuclear intensity value. The second statistical nuclear size value may be less than the third statistical nuclear size value. The relationship of the cell types and nuclear size and nuclear intensity may be similar to the illustration in FIG. 2E.

At block 2450, statistical feature values for one or more properties of the cells of the cell types may be determined. The statistical feature values may include mean, median, mode, or percentile of the one or more properties of the cells of the cell types. Determining the statistical feature values for the one or more properties of the cells of the cell types may include determining a statistical feature value for each of the one or more properties for each cell type of the plurality of cell types. For example, if there are x cell types and y properties, then there are x×y statistical feature values.

The one or more properties may include a density of the cell type and a ratio of the cell type and other cell types. For example, the one or more properties may include CLL-like cell ratio, aCLL-like cell ratio, RT-like cell ratio, CLL-like cell density, aCLL-like cell density, and/or RT-like cell density, including any combinations thereof. The ratio may be the ratio of the cell type to all cell types. In some embodiments, the ratio may be the ratio of the cell type to other cell types.

At block 2460, a classification of leukemia or lymphoma in the subject is determined using the statistical feature values for the one or more properties of each of the plurality of cell types. The properties may be any properties described herein. Determining the classification of leukemia or lymphoma in the subject may include using no more than 1, 2, 3, 4, 5, or 6 statistical feature values for the one or more properties. In some embodiments, the properties may include CLL-like cell density. In some embodiments, the properties may include cell densities (e.g., CLL-like cell density, aCLL-like cell density, RT-like cell density) and/or may exclude cell ratios. In some embodiments, the classification may be determined without cell morphologic or cytologic traits other using nuclear size and nuclear intensity to classify cell types.

The classification may be of Hodgkin's lymphoma or non-Hodgkin's lymphoma. Determining the classification of leukemia or lymphoma may include determining a level of chronic lymphocytic leukemia (CLL), accelerated CLL, or Richter transformation (RT). In some embodiments, the classification may be determining a level of a normal, healthy condition (i.e., without leukemia or lymphoma). In some embodiments, the classification is of Hodgkin's lymphoma (e.g., presence or different severity or levels of Hodgkin's lymphoma). The classification of leukemia or lymphoma may be that the subject has leukemia or lymphoma. For example, the classification may be that any of CLL, aCLL, or RT exists, and leukemia or lymphoma may be determined to exist. In some embodiments, the classification of a disease may be determined in combination with considering clinical data of the subject, including symptoms of lymphoma.

Process 2400 may include comparing the classification with a previous classification of leukemia or lymphoma in the subject and determining a progression of the leukemia or lymphoma in the subject based on the comparing. The previous classification may be determined using process 2400.

The classification of leukemia or lymphoma may be that the subject has leukemia or lymphoma. Process 2400 may include treating the subject. Treatment may include any treatment described herein, including treatments described with process 700.

Determining the classification may use a machine learning model that is trained using supervised learning. The plurality of lymphoid cells may be a first plurality of lymphoid cells. The statistical feature values may be first statistical feature values. The machine learning model may be trained by receiving a plurality of training images. Each training image may include a second plurality of lymphoid cells. Each lymphoid cell of the second plurality of lymphoid cells may be classified into one of the plurality of cell types. Each training image of the plurality of training images may be labeled with a known classification of leukemia or lymphoma. For each training image of the plurality of training images, second statistical feature values may be determined for the one or more properties of each of the plurality of cell types of the respective second plurality of lymphoid cells. Parameters of the machine learning model may be optimized based on outputs of the machine learning model matching or not matching the known classification when the second statistical feature values are input into the machine learning model. An output of the machine learning model specifies a classification of leukemia or lymphoma for the training image. In some embodiments, process 2400 may include training the machine learning model using the plurality of training images.

The machine learning model may include various supervised learning models, such as a decision tree model, which may be boosted, e.g., using XGBoost. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm. The machine learning model may also include other machine learning models, including convolutional neural networks (CNN), linear regression, logistic regression, deep recurrent neural network (e.g., long short-term memory, LSTM), Bayes classifier, hidden Markov model (HMM), linear discriminant analysis (LDA), random forest algorithm, and support vector machine (SVM). The machine learning model may be any model described herein.

Process 2400 may include additional implementations, such as any single implementation or any combination of implementations described herein and/or in connection with one or more other processes described elsewhere herein.

Although FIG. 24 shows example blocks of process 2400, in some implementations, process 2400 may include additional blocks, fewer blocks, different blocks, or differently arranged blocks than those depicted in FIG. 24. Additionally, or alternatively, two or more of the blocks of process 2400 may be performed in parallel.

### III. Example Measurement Systems

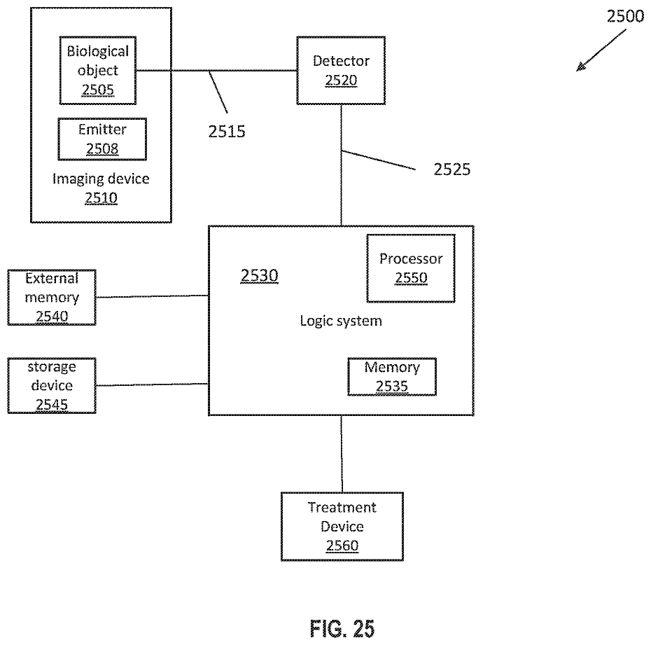

FIG. 25 illustrates a measurement system 2500 according to an embodiment of the present disclosure. The system as shown includes a biological object 2505, such as a region of an organism (e.g., human), within an imaging device 2510, where an emitter 2508 can send waves to biological object 2505. For example, biological object 2505 can receive magnetic fields and/or radio waves from emitter 2508 to provide a signal of a physical characteristic 2515. Biological object 2505 may include objects treated with a contrast agent or other agent to facilitate imaging. An example of an imaging device can be a microscope. Imaging device 2510 may include multiple modules.

Physical characteristic 2515 (e.g., an optical intensity, a voltage, or a current), from the biological object is detected by detector 2520. Detector 2520 can take a measurement at intervals (e.g., periodic intervals) to obtain data points that make up a data signal. In one embodiment, an analog-to-digital converter converts an analog signal from the detector into digital form at a plurality of times. Imaging device 2510 and detector 2520 can form an assay system, e.g., a microscope system that acquires image data according to embodiments described herein. A data signal 2525 is sent from detector 2520 to logic system 2530. As an example, data signal 2525 can be used to determine locations of particular objects (e.g., organs, tissues, implanted material) in a biological object. Data signal 2525 can include various measurements made at a same time, e.g., different signals for different areas of biological object 2505, and thus data signal 2525 can correspond to multiple signals. Data signal 2525 may be stored in a local memory 2535, an external memory 2540, or a storage device 2545.

Logic system 2530 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2530 and the other components may be part of a stand-alone or network connected computer system, or they may be directly attached to or incorporated in a device (e.g., an imaging system) that includes detector 2520 and/or imaging device 2510. Logic system 2530 may also include software that executes in a processor 2550. Logic system 2530 may include a computer readable medium storing instructions for controlling measurement system 2500 to perform any of the methods described herein. For example, logic system 2530 can provide commands to a system that includes imaging device 2510 such that magnetic emission or other physical operations are performed.

Measurement system 2500 may also include a treatment device 2560, which can provide a treatment to the subject. Treatment device 2560 can determine a treatment and/or be used to perform a treatment. Examples of such treatment can include surgery, radiation therapy, chemotherapy, immunotherapy, targeted therapy, hormone therapy, stem cell transplant, and implantation of radioactive seeds. Logic system 2530 may be connected to treatment device 2560, e.g., to provide results of a method described herein. The treatment device may receive inputs from other devices, such as an imaging device and user inputs (e.g., to control the treatment, such as controls over a robotic system).

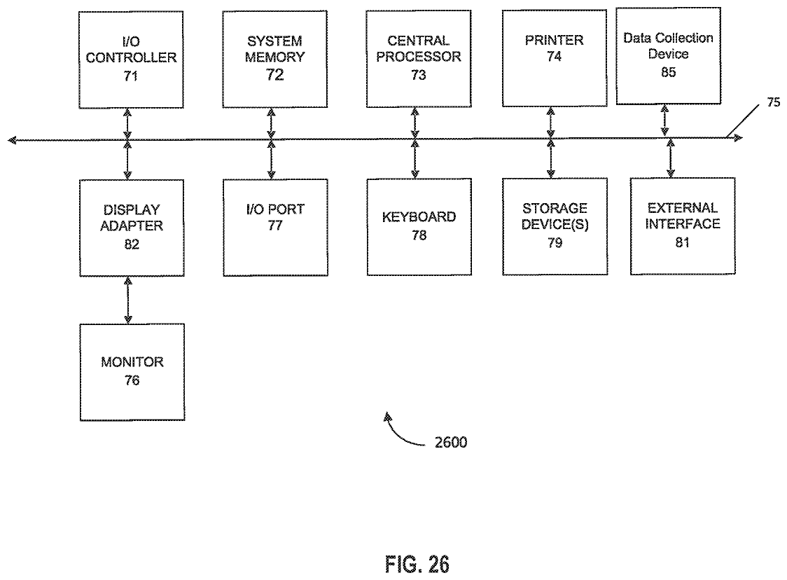

Any of the computer systems mentioned herein may utilize any suitable number of subsystems. Examples of such subsystems are shown in FIG. 26 in computer system 2600. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 26 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning). For example, I/O port 77 or external interface 81 (e.g., Ethernet, Wi-Fi, etc.) can be used to connect computer system 2600 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

1. A method for analyzing a plurality of lymphoid cells, the method comprising:
receiving data of an image captured of the plurality of lymphoid cells in a biological sample from a subject, the data including pixels,
identifying, in the data, first pixels corresponding to nuclei of the plurality of lymphoid cells;
measuring a value for nuclear size, a value for nuclear intensity, and a value for cellular density using the first pixels; and
determining, using the measured values, a classification of leukemia or lymphoma in the subject.

2. A method for analyzing a plurality of lymphoid cells, the method comprising:
receiving data of an image captured of the plurality of lymphoid cells in a biological sample from a subject, wherein the data includes pixels;
identifying, in the data, first pixels corresponding to the plurality of lymphoid cells;
segmenting, using the first pixels, each lymphoid cell of the plurality of lymphoid cells into second pixels corresponding to a first portion with a nucleus and third pixels corresponding to a second portion without a nucleus;
filtering, using the second pixels and the third pixels, the plurality of lymphoid cells to remove lymphoid cells having overlapping first portions to produce fourth pixels corresponding to a filtered plurality of lymphoid cells;
measuring a value for nuclear size, a value for nuclear intensity, and a value for cellular density using the fourth pixels corresponding to the filtered plurality of lymphoid cells; and
determining, using the measured values, a classification of leukemia or lymphoma in the subject.

3. The method of claim 1 or 2, further comprising measuring a value for distances between cells, wherein the measured values comprise the value for distances between cells.

4. The method of claim 3, wherein measuring the value for distances between cells comprises measuring a value for distances between the nuclei.

5. The method of claim 4, wherein measuring the value for distances between the nuclei comprises:
determining a centroid for each nucleus of each lymphoid cell of the filtered plurality of lymphoid cells, and
measuring a value for distances between centroids.

6. The method of claim 3, wherein measuring the value for distances between cells comprises measuring the distances between each cell and the nearest neighbor of each cell.

7. The method of claim 3, wherein determining the classification of leukemia or lymphoma uses no values measured from the filtered plurality of lymphoid cells other than the value for nuclear size, the value for nuclear intensity, the value for cellular density, and the value for distances between cells.

8. The method of claim 1 or 2, wherein determining the classification of leukemia or lymphoma comprises determining a level of chronic lymphocytic leukemia (CLL), accelerated CLL, or Richter transformation (RT).

9. The method of claim 1 or 2, wherein the classification is of Hodgkin's lymphoma or non-Hodgkin's lymphoma.

10. The method of claim 1 or 2, wherein measuring the value for nuclear intensity comprises:
measuring subvalues for a plurality of color channels, and
determining a statistical value of the subvalues for the plurality of color channels.

11. The method of claim 1 or 2, wherein measuring the value for nuclear size comprises:
for each lymphoid cell of the filtered plurality of lymphoid cells:
measuring a cellular value for the nuclear size,
comparing the cellular value to a cutoff value, and
determining whether the lymphoid cell is in a size classification using the comparison; and
calculating an amount of lymphoid cells in the size classification.

12. The method of claim 11, wherein the amount of lymphoid cells in the size classification is a ratio determined using a number of lymphoid cells in the size classification and a number of lymphoid cells not in the size classification.

13. The method of claim 11, wherein:
the cutoff value is determined using an objective function, and
the objective function determined using amounts of lymphoid cell types in a training data set.

14. The method of claim 1 or 2, wherein the plurality of lymphoid cells comprises at least 1,000 lymphoid cells.

15. The method of claim 1 or 2, wherein filtering the plurality of lymphoid cells comprises:
for each lymphoid cell of the plurality of lymphoid cells:
determining a ratio of the first portion with the nucleus and the second portion without the nucleus,
comparing the ratio to a threshold value, and
determining the lymphoid cell is in the filtered plurality of lymphoid cells when the ratio exceeds the threshold value.

16. The method of claim 1 or 2, wherein:
the plurality of lymphoid cells is a first plurality of lymphoid cells,
the value for nuclear size is a first value for nuclear size,
the value for nuclear intensity is a first value for nuclear intensity,
the value for cellular density is a first value for cellular density, and
determining the classification uses a machine learning model trained by:
receiving a plurality of training images, each training image comprising a second plurality of lymphoid cells, and each training image of the plurality of training images labeled with a known classification of leukemia or lymphoma;
for each training image of the plurality of training images:
measuring a second value for nuclear size, a second value for nuclear intensity, and a second value for cellular density;
optimizing parameters of the machine learning model based on outputs of the machine learning model matching or not matching the known classification when the second values are input into the machine learning model, wherein an output of the machine learning model specifies a classification of leukemia or lymphoma for the training image.

17. The method of claim 16, wherein the machine learning model comprises a convolutional neural network.

18. The method of claim 16, further comprising training the machine learning model using the plurality of training images.

19. The method of claim 1 or 2, wherein the classification of leukemia or lymphoma is that the subject has leukemia or lymphoma, the method further comprising treating the subject.

20. The method of claim 1 or 2, further comprising:
comparing the classification with a previous classification of leukemia or lymphoma in the subject, and
determining a progression of the leukemia or lymphoma in the subject based on the comparing.

21. The method of claim 1 or 2, wherein the data of the image comprises at least 100,000 pixels.

22. The method of claim 1 or 2, further comprising capturing the image of the plurality of lymphoid cells in the biological sample by performing microscopy on the biological sample.

23. The method of claim 22, further comprising obtaining the biological sample from the subject.

24. The method of claim 1 or 2, wherein:
the plurality of lymphoid cells is a first plurality of lymphoid cells, and
the image comprises a second plurality of lymphoid cells,
the method further comprising:
selecting a region comprising the first plurality of lymphoid cells as representative of the biological sample from the subject prior to segmenting each lymphoid cell of the first plurality of lymphoid cells.

25. A method for analyzing a plurality of lymphoid cells, the method comprising:
receiving data of an image captured of the plurality of lymphoid cells in a biological sample from a subject, wherein the data includes pixels;
identifying, in the data, first pixels corresponding to the plurality of lymphoid cells;
segmenting, using the first pixels, each lymphoid cell of the plurality of lymphoid cells into second pixels corresponding to a first portion with a nucleus and third pixels corresponding to a second portion without a nucleus;
filtering, using the second pixels and the third pixels, the plurality of lymphoid cells to remove lymphoid cells having overlapping first portions to produce fourth pixels corresponding to a filtered plurality of lymphoid cells;
measuring a value for nuclear size and a value for nuclear intensity for each lymphoid cell of the filtered plurality of lymphoid cells using the fourth pixels;
determining a value of a parameter using the measured values;
comparing the value of the parameter to a reference value; and
determining, using the comparison, a classification of leukemia or lymphoma in the subject.

26. The method of claim 25, wherein the value of the parameter is determined using the formula:
$$\frac{1}{{N}_{nucleus}} {\sum }_{i = 1}^{{N}_{nucleus}} \sqrt{S ( {nucleus}_{i} ) * {I}_{mean} ( {nucleus}_{i} )}$$
where:
N_{nucleus} is the number of nuclei,
i is an integer,
S(nucleusᵢ) is the size of nucleusᵢ, and
Iₘₑₐₙ(nucleusᵢ) is the mean intensity of nucleusᵢ.

27. A method for analyzing a plurality of lymphoid cells, the method comprising:
receiving data of an image captured of the plurality of lymphoid cells in a biological sample from a subject, wherein the data includes a plurality of pixels;
identifying, in the data, first pixels corresponding to the plurality of lymphoid cells, the plurality of pixels comprising the first pixels;
segmenting, using the first pixels, each lymphoid cell of the plurality of lymphoid cells into second pixels corresponding to a first portion with a nucleus and third pixels corresponding to a second portion without a nucleus;
filtering, using the second pixels and the third pixels, the plurality of lymphoid cells to remove lymphoid cells having overlapping first portions to produce fourth pixels corresponding to a filtered plurality of lymphoid cells;
measuring a value for nuclear size and a value for nuclear intensity for each lymphoid cell of the filtered plurality of lymphoid cells using the fourth pixels;
dividing the data of the image into a plurality of tiles;
for each tile of the plurality of tiles:
determining a value of a parameter using the measured values of nuclear size and the measured values of nuclear intensity for lymphoid cells corresponding to the fourth pixels in the tile; and
identifying, using the values of the parameter, a subset of the plurality of tiles as representing cells with increased mitotic activity.

28. The method of claim 27, wherein identifying the subset of the plurality of tiles comprises:
for each tile of the plurality of tiles:
comparing the value of the parameter with a threshold value, and
determining the tile to be in the subset when the value exceeds the threshold value.

29. The method of claim 28, wherein the threshold value is determined by Otsu's method.

30. The method of claim 27, further comprising:
determining a classification of leukemia or lymphoma in the subject using the subset of the plurality of tiles.

31. The method of claim 30, wherein determining the classification of leukemia or lymphoma in the subject comprises:
determining a statistical value of the values of the parameter for the subset of the plurality of tiles, and
comparing the statistical value to a cutoff value.

32. The method of claim 30, wherein determining the classification of leukemia or lymphoma in the subject comprises:
determining an amount of tiles in the subset of the plurality of tiles relative to the plurality of tiles, and
comparing the amount to a cutoff amount.

33. The method of claim 30, wherein determining the classification of leukemia or lymphoma comprises determining a level of chronic lymphocytic leukemia (CLL), accelerated CLL, or Richter transformation (RT).

34. The method of claim 27, further comprising:
generating an image comprising fifth pixels, wherein:
the intensity of each fifth pixel of the fifth pixels is determined using the value of the parameter for a tile of the subset of the plurality of tiles.

35. The method of claim 27 or 34, further comprising:
determining a shape classification of the subset of the plurality of tiles,
determining a classification of leukemia or lymphoma in the subject using the shape classification.

36. A method for analyzing a plurality of lymphoid cells, the method comprising:
receiving data of an image captured of the plurality of lymphoid cells in a biological sample from a subject, wherein the data includes a plurality of pixels;
identifying, in the data, first pixels corresponding to the plurality of lymphoid cells, the plurality of pixels comprising the first pixels;
segmenting, using the first pixels, each lymphoid cell of the plurality of lymphoid cells into second pixels corresponding to a first portion with a nucleus and third pixels corresponding to a second portion without a nucleus;
filtering, using the second pixels and the third pixels, the plurality of lymphoid cells to remove lymphoid cells having overlapping first portions to produce fourth pixels corresponding to a filtered plurality of lymphoid cells;
measuring a value for nuclear size and a value for nuclear intensity for each lymphoid cell of the filtered plurality of lymphoid cells using the fourth pixels;
dividing the data of the image into a plurality of tiles;
for each tile of the plurality of tiles:
determining a value of a parameter using the measured values of nuclear size and the measured values of nuclear intensity for lymphoid cells corresponding to the fourth pixels in the tile; and
determining a classification of leukemia or lymphoma using the values of the parameters for the plurality of tiles.

37. The method of claim 36, wherein determining the classification of leukemia or lymphoma in the subject comprises:
determining a statistical value of the values of the parameter for the subset of the plurality of tiles, and
comparing the statistical value to a cutoff value.

38. The method of claim 36, wherein:
the plurality of lymphoid cells is a first plurality of lymphoid cells,
the value of the parameter is a first value of the parameter,
the plurality of tiles is a first plurality of tiles, and
determining the classification of leukemia or lymphoma uses a machine learning model trained by:
receiving a plurality of training images, each training image comprising a second plurality of lymphoid cells, and each training image of the plurality of training images labeled with a known classification of leukemia or lymphoma;
for each training image of the plurality of training images:
dividing the training image into a plurality of second tiles, and
determining a second value of the parameter for each tile of the plurality of second tiles;
optimizing parameters of the machine learning model based on outputs of the machine learning model matching or not matching the known classification when the second values for each training image are input into the machine learning model, wherein an output of the machine learning model specifies a classification of leukemia or lymphoma for the training image.

39. The method of claim 38, wherein optimizing the parameters of the machine learning model comprises inputting location values corresponding to the second values into the machine learning model.

40. The method of claim 27 or 38, further comprising:
for each tile of the plurality of tiles:
determining a number of lymphoid cells in the tile,
wherein the value of the parameter is determined using the formula:
$$\frac{1}{{N}_{nucleus}} {\sum }_{i = 1}^{{N}_{nucleus}} \sqrt{S ( {nucleus}_{i} ) * {I}_{mean} ( {nucleus}_{i} )}$$
where:
N_{nucleus} is the number of lymphoid cells,
i is an integer,
S(nucleusᵢ) is the size of nucleusᵢ, and
Iₘₑₐₙ(nucleusᵢ) is the mean intensity of nucleusᵢ.

41. The method of claim 27 or 38, wherein the tiles of the plurality of tiles are the same size.

42. The method of claim 27 or 38, wherein each tile of the plurality of tiles overlaps with at least one other tile of the plurality of tiles.

43. A method for analyzing a plurality of lymphoid cells, the method comprising:
receiving data of an image captured of the plurality of lymphoid cells in a biological sample from a subject, the data including pixels;
identifying, in the data, first pixels corresponding to nuclei of a set of the plurality of lymphoid cells, the set satisfying one or more criteria;
for each cell of the set of the plurality of lymphoid cells, measuring a value for nuclear size and a value for nuclear intensity using the first pixels;
classifying each lymphoid cell of the set of the plurality of lymphoid cells into one of a plurality of cell types using the value for nuclear size of the cell and the value for nuclear intensity of the cell;
determining statistical feature values for one or more properties of the cells of the cell types; and
determining, using the statistical feature values for the one or more properties of each of the plurality of cell types, a classification of leukemia or lymphoma in the subject.

44. The method of claim 43, wherein identifying the first pixels corresponding to the nuclei of set of the plurality of lymphoid cells comprises:
identifying, in the data, second pixels corresponding to the plurality of lymphoid cells;
segmenting, using the second pixels, each lymphoid cell of the plurality of lymphoid cells into third pixels corresponding to a first portion with a nucleus and fourth pixels corresponding to a second portion without a nucleus; and
filtering, using the third pixels and the fourth pixels, the plurality of lymphoid cells to remove lymphoid cells having overlapping first portions to produce the first pixels.

45. The method of claim 43, wherein the one or more criteria comprise a threshold nuclear size value, a threshold nuclear intensity value, a threshold value for area to perimeter, or a threshold solidity value.

46. The method of claim 43, wherein:
the plurality of lymphoid cells is a first plurality of lymphoid cells, and
the image comprises a second plurality of lymphoid cells,
the method further comprising:
selecting a region comprising the first plurality of lymphoid cells as representative of the biological sample from the subject prior to identifying the first pixels.

47. The method of claim 43, wherein the plurality of cell types comprises chronic lymphocytic leukemia-like (CLL-like) cells, accelerated CLL-like (aCLL-like) cells, and Richter transformation-like (RT-like) cells.

48. The method of claim 47, wherein:
the CLL-like cells are characterized by a first statistical nuclear size value for and a first statistical nuclear intensity value,
the aCLL-like cells are characterized by a second statistical nuclear size value and a second statistical nuclear intensity value,
the RT-like cells are characterized by a third statistical nuclear size value and a third statistical nuclear intensity value,
the first statistical nuclear size value is less than the third statistical nuclear size value,
the first statistical nuclear intensity value is less than the second statistical nuclear intensity value and the third statistical nuclear intensity value, and
the second statistical nuclear size value is less than the third statistical nuclear size value.

49. The method of claim 43, wherein determining the statistical feature values for the one or more properties of the cells of the cell types comprises:
for each cell type of the plurality of cell types, determining a statistical feature value for each of the one or more properties of the cells of the cell type.

50. The method of claim 43, wherein determining the classification of leukemia or lymphoma in the subject comprises using no more than 6 statistical feature values for the one or more properties.

51. The method of claim 43, wherein the one or more properties comprise a density of the cell type and a ratio of the cell type and other cell types.

52. The method of claim 51, wherein the one or more properties comprise CLL-like cell ratio, aCLL-like cell ratio, RT-like cell ratio, CLL-like cell density, and aCLL-like cell density.

53. The method of claim 51, wherein the ratio is of the cell type to all cell types.

54. The method of claim 52, wherein the one or more properties further comprise RT-like cell density.

55. The method of claim 43, wherein the statistical feature values comprise mean, median, mode, or percentile of the one or more properties of the cells of the cell types.

56. The method of claim 43, wherein classifying each lymphoid cell of the set of the plurality of lymphoid cells comprises unsupervised clustering.

57. The method of claim 43, wherein:
the plurality of lymphoid cells is a first plurality of lymphoid cells,
the statistical feature values are first statistical feature values, and
determining the classification uses a machine learning model trained by:
receiving a plurality of training images, each training image comprising a second plurality of lymphoid cells, each lymphoid cell of the second plurality of lymphoid cells classified into one of the plurality of cell types, and each training image of the plurality of training images labeled with a known classification of leukemia or lymphoma;
for each training image of the plurality of training images:
determining second statistical feature values for the one or more properties of each of the plurality of cell types of the respective second plurality of lymphoid cells;
optimizing parameters of the machine learning model based on outputs of the machine learning model matching or not matching the known classification when the second statistical feature values are input into the machine learning model, wherein an output of the machine learning model specifies a classification of leukemia or lymphoma for the training image.

58. The method of claim 57, wherein the machine learning model comprises a decision tree model.

59. The method of claim 57, further comprising training the machine learning model using the plurality of training images.

60. The method of claim 43, wherein the set of the plurality of lymphoid cells comprise at least 500, 1,000, 5,000, 10,000, or 20,000 lymphoid cells.

61. The method of claim 43, wherein determining the classification of leukemia or lymphoma comprises determining a level of chronic lymphocytic leukemia (CLL), accelerated CLL, or Richter transformation (RT).

62. The method of claim 43, wherein the classification is of Hodgkin's lymphoma or non-Hodgkin's lymphoma.

63. The method of claim 43, wherein measuring the value for nuclear intensity comprises:
measuring subvalues for a plurality of color channels, and
determining a statistical value of the subvalues for the plurality of color channels.

64. The method of claim 43, wherein the classification of leukemia or lymphoma is that the subject has leukemia or lymphoma, the method further comprising treating the subject.

65. The method of claim 43, further comprising:
comparing the classification with a previous classification of leukemia or lymphoma in the subject, and
determining a progression of the leukemia or lymphoma in the subject based on the comparing.

66. The method of claim 43, wherein the data of the image comprises at least 100,000 pixels.

67. The method of claim 43, further comprising capturing the image of the plurality of lymphoid cells in the biological sample by performing microscopy on the biological sample.

68. The method of claim 67, further comprising obtaining the biological sample from the subject.

69. A computer product comprising a non-transitory computer readable medium storing a plurality of instructions that when executed control a computer system to perform the method of any one of the preceding claims.

70. A system comprising:
the computer product of claim 69; and
one or more processors for executing instructions stored on the computer readable medium.

71. A system comprising means for performing any of the above methods.

72. A system comprising one or more processors configured to perform any of the above methods.

73. A system comprising modules that respectively perform the steps of any of the above methods.
